(19) United States
(12) Patent Application Publication
(10) Pub. No.: US 2024/0271102 A1
(43) Pub. Date: Aug. 15, 2024

(54) METHODS FOR ENGINEERING AND USE OF CILIATED ORGANOIDS HAVING NATIVE-LIKE, APICAL-OUT POLARITY

(71) Applicant: Carnegie Mellon University, Pittsburgh, PA (US)

(72) Inventors: Xi Ren, Wexford, PA (US); Piyumi Wijesekara, Pittsburgh, PA (US); Amir Barati Farimani, Pittsburgh, PA (US); Prakarsh Yadav, Pittsburgh, PA (US)

(21) Appl. No.: 18/568,091

(22) PCT Filed: Jun. 8, 2022
(86) PCT No.: PCT/US2022/072815

§ 371 Date: Dec. 7, 2023

Related U.S. Application Data
(60) Provisional application No. 63/274,126, filed on Nov. 1, 2021.
(60) Provisional application No. 63/208,201, filed on Jun. 8, 2021.

(51) Int. Cl. C12N 5/071; C12M 1/32; G01N 33/50

(52) U.S. Cl. CPC C12N 5/0688; C12M 23/12; G01N 33/5005; C12N 2500/02; C12N 2501/727; C12N 2513/00

(57) ABSTRACT

Methods and materials for making and using apical-out ciliated organoids (e.g., apical-out airway organoids) are provided herein.

### CROSS REFERENCE TO RELATED APPLICATIONS

This application claims benefit of priority under 35 U.S.C. § 119(e) to U.S. Provisional Application No. 63/208,201, filed on Jun. 8, 2021, and U.S. Provisional Application No. 63/274,126, filed on Nov. 1, 2021, the entire contents of which are hereby incorporated by reference.

### STATEMENT AS TO FEDERALLY SPONSORED RESEARCH

This invention was made with United States government support under W81XWH-21-1-0183 awarded by U.S. ARMY MEDICAL RESEARCH ACQUISITION ACTIVITY (USAMRAA). The U.S. government has certain rights in the invention.

### TECHNICAL FIELD

This document relates to methods and materials for making and using apical-out ciliated organoids, including apical-out airway organoids.

### BACKGROUND

Organoids are three-dimensional (3D) in vitro cell cultures that incorporate at least some of key features of an organ. In general, organoids contain organ-specific cell types that are spatially self-organized in a manner similar to what is observed in vivo. In addition, organoid cells typically can recapitulate at least some functions of the represented organ. See, for example, Sato and Clevers, Science. 340(6137):1190-1194, 2013; Wells and Spence, Development. 141(4):752-760, 2014; Lancaster and Knoblich, Science. 345(6194):1247125, doi: 10.1126/science.1247125, 2014; and Huch and Koo, Development. 142(18):3113-3125, 2015.

The human conducting airway is composed of polarized pseudostratified epithelium, with cilia movement and mucus secretion taking place on the apical side that faces the external environment and directly interacts with respiratory pathogens. Polarized organoids can be generated using airway basal epithelial cells. However, conventional airway organoids engineered from airway basal cells in extracellular matrix-embedded culture have an apical-in conformation, which makes the apical surface difficult to access and precludes effective investigation of airway pathology in a physiologically relevant manner. Moreover, motile cilia project from the airway apical surface and directly interface with inhaled external environment, but the apical-in conformation of prevailing airway organoid models results in cilia facing the organoid interior. This positioning, in addition to cilia's nanoscale dimension and high beating frequency, renders quantitative assessment of cilia motility a sophisticated and challenging task.

### SUMMARY

The methods described herein include approaches to engineering apical-out, ciliated organoids (e.g., airway organoids) by culturing basal cell clusters in suspension without any extracellular matrix support, which can effectively reverse the organoid polarity from that achieved using standard technologies. Accordingly, the apical-out organoids generated using methods provided herein offer unique advantages over conventional apical-in organoids for investigating lung biology, respiratory infection, and other respiratory pathology.

This document provides methods and materials for reproducible engineering of apical-out organoids (e.g., apical-out airway organoids, also referred to as AOAOs) of defined size, with consistent physical characteristics from one preparation to another (provided that the same method steps are used for each preparation). As described herein, for example, mature AOAO exhibited stable rotational motion propelled by the beating of exterior-facing cilia when surrounded by MATRIGEL® (a solubilized basement membrane matrix secreted by Engelbreth-Holm-Swarm mouse sarcoma cells). Also as described herein, a computational framework leveraging computer vision algorithms was developed to quantify AOAO rotation, and the correlation of the framework with direct measurement of cilia motility was validated. In addition, the feasibility of using AOAO rotation to recapitulate and measure defective cilia motility caused by chemotherapy-induced toxicity and by CCDC39 mutations in cells from primary ciliary dyskinesia patients was established. The rotating AOAO model and the associated computational pipeline provides a generalizable framework that can be adopted to develop high throughput assays in order to expedite modeling of and therapeutic development for genetic and environmental ciliopathies.

Thus, this document provides methods and materials for generating and using ciliated apical-out organoids (e.g., AOAOs, including human AOAOs). As described herein, the ciliated apical-out organoids offer unique advantages over conventional apical-in organoids for investigating lung biology and respiratory pathology.

In a first aspect, this document features a method for determining whether an agent has an effect on a ciliated organoid having apical-out polarity. The method can include, or consist essentially of, contacting a ciliated organoid with an agent, comparing a characteristic of the organoid after the contacting with the same characteristic of the organoid before the contacting, and when the characteristic after the contacting has changed as compared to the characteristic before the contacting, determining that the agent has an effect on the organoid, or when the characteristic after the contacting has not changed as compared to the characteristic before the contacting, determining that the agent does not have an effect on the organoid. The ciliated organoid can contain airway epithelial cells, fallopian tube epithelial cells, middle ear epithelial cells, brain ventricular epithelial cells, or any combination thereof. The ciliated organoid can be an apical-out airway organoid (AOAO) that contains normal human bronchial epithelial cells (NHBEs), tracheal epithelial cells, nasal epithelial cells, or any combination thereof. The ciliated organoid can further contain stromal cells, vascular endothelial cells, immune cells, or any combination thereof. The characteristic can be coordinated percentage ciliation, ciliary beating, goblet cell specification, organoid rotation, organoid angular velocity, organoid locomotion in two dimensions, mucus secretion, cytokine secretion, extracellular matrix secretion, cell viability, or cell death. For example, the characteristic can be coordinated ciliary beating. For example, characteristic can be organoid rotation in three dimensions. For example, the characteristic can be organoid locomotion in two dimensions. The agent can be a therapeutic agent, a pathogen, a pollutant, or a chemical or biological agent (e.g., a cytokine or a cytotoxic reagent). The agent can be radiation. The method can include determining the characteristic about 1 hour to about 28 days after the contacting.

In another aspect, this document features a method for producing a ciliated organoid having apical-out polarity. The method can include, or consist essentially of, suspending a plurality of epithelial cells in a differentiation medium supplemented with a cytoskeletal structure modulator, wherein the medium does not contain extracellular matrix components, placing an aliquot of the suspended cells into one or more wells of a cell-repellent microplate, and maintaining the microplate for 14 to 28 days under conditions such that the suspended cells aggregate and differentiate to form a ciliated organoid with apical-out polarity. The epithelial cells can include airway basal cells, fallopian tube epithelial cells, middle ear epithelial cells, brain ventricular epithelial cells, or any combination thereof. For example, the epithelial cells can be airway basal cells, and the airway basal cells can include normal human bronchial epithelial cells (NHBEs), airway bronchial stem cells (ABSCs), tracheal epithelial cells, nasal epithelial cells, or any combination thereof. The method can further include adding stromal cells, vascular endothelial cells, immune cells, or any combination thereof to the plurality of epithelial cells. The differentiation medium can be is an air-liquid interface (ALI) medium. The cytoskeletal structure modulator can be a Rho-associated kinase (ROCK) inhibitor (e.g., Y27632). The aliquot can contain about 50 to 5000 cells. The cell-repellant microplate can be a 96-well plate (e.g., a 96-well plate having U-shaped wells, or a 96-well plate having V-shaped wells). The cell-repellant microplate can be a 12-well, 24-well, 48-well, 384-well, or 1536-well plate. The conditions can include a temperature of about 37° C. and an atmosphere containing 5% CO₂.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A is a schematic depicting the generation of a traditional apical-in airway organoid in hydrogel culture. FIG. 1B is a schematic depicting the engineering of an apical-out airway organoid from a basal cell cluster in suspension culture. The apical-out organoid allows biomimetic epithelial interaction with respiratory pathogens (top right). The beating of cilia in the apical-out organoid leads to organoid rotation in suspension. (bottom right).

FIG. 2 is an image showing Normal Human Bronchial Epithelial cells (NHBEs) at passage 2 of culture in Bronchial Epithelial Cell Growth Basal Medium (BEGM™) with added small molecule factors.

FIGS. 3A-3F are images showing cell spheroids formed on day 1 on a U-bottom cell repellent surface with 1000 (FIG. 3A), 500 (FIG. 3B), 200 (FIG. 3C), 100 (FIG. 3D), 50 (FIG. 3E), and 20 (FIG. 3F) NHBEs in differentiation medium.

FIGS. 4A-4F are images showing cell assemblies formed on day 1 on a U-bottom cell repellent surface with 1000 (FIG. 4A), 500 (FIG. 4B), 200 (FIG. 4C), 100 (FIG. 4D), 50 (FIG. 4E), and 20 (FIG. 4F) NHBEs in differentiation medium and 10 μM Y27632.

FIGS. 5A-5C are images showing cell spheroid formation at day 2 on a U-bottom cell repellent surface in differentiation medium (FIG. 5A), BEGM (FIG. 5B), and BEGM with added small molecule factors (FIG. 5C).

FIGS. 6A-6C are images showing cell spheroid formation at day 2 on a U-bottom cell repellent surface with 125 RPM rotation in differentiation medium (FIG. 6A), BEGM (FIG. 6B), and BEGM with added small molecule factors (FIG. 6C).

FIG. 7 is a graph plotting the percentage of differentiated organoids that rotated when embedded in the indicated supporting matrices. ***, p<0.001; **, p<0.01.

FIG. 8 includes images indicating the cellular composition of Day 1 organoids, via whole mount immunofluorescence staining of organoids for the airway basal epithelial cell marker p63.

FIGS. 9A and 9B show a time-course analysis of ciliated cell expression via the ciliated nuclear marker FOXJ1. FIG. 9A includes immunofluorescent images of organoids stained for FOXJ1 at Days 1, 3, 7, 14, and 21. FIG. 9B is a graph plotting quantified ciliated cell expression.

FIG. 10 includes a pair of immunofluorescent images of apical-out organoids stained for the cilia marker, acetylated alpha tubulin.

FIGS. 11A and 11B show a time-course analysis of expression of the epithelial tight junction protein ZO-1. FIG. 11A includes immunofluorescent images of organoids stained for ZO-1 at Days 1, 3, 7, and 21. FIG. 11B is an image showing a cross section of a Day 21 apical-out organoid stained for ZO-1.

FIGS. 12A and 12B are images depicting the apical-out phenotype of an organoid via scanning electron microscopy (SEM; FIG. 12A) and transmission electron microscopy (TEM; FIG. 12B).

FIGS. 13A and 13B are images showing apical-out organoids stained for goblet cell marker MUC5AC after treatment with 1 ng/mL IL-13 (FIG. 13A) or without IL-13 treatment (FIG. 13B).

FIGS. 14A-14H show characterization of engineered AOAOs. FIG. 14A is a diagram of apical-in versus apical-out airway organoids. FIG. 14B includes immunofluorescence images of Day 21 AOAOs stained with markers of cilia (Ac-α-Tub) and tight junction (ZO-1). Scale bar, 25 μm. FIG. 14C includes SEM (scale bar, 10 μm) and TEM (scale bar, 800 nm (left), 400 nm (right)) images of AOAOs. FIG. 14D is a graph plotting the percentage of Day 21 (D21) organoids with apical-out versus apical-in epithelial polarity indicated by apical Ac-α-Tub localization. FIG. 14E includes images showing a time-series characterization of AOAO maturation by immunostaining of FOXJ1 (a nuclear marker of ciliated cells), Ac-α-Tub, and ZO-1. Scale bar, 25 μm. FIG. 14F is a diagram depicting an approach for assessing percentage ciliation by quantifying surface coverage of Ac-α-Tub expression. FIGS. 14G and 14H are graphs plotting time-series quantification of FOXJ1+ cell abundance (FIG. 14G) and percentage ciliation (FIG. 14H) in AOAOs. All data represent means±s.d. from ≥15 organoids. ***, p<0.00¹.

FIGS. 15A and 15B show airway organoid formation in different medium in 3D suspension culture. FIG. 15A is a diagram showing the conditions under comparison: during Day 0 to Day 1 of 3D suspension culture, where the organoid was either cultured in BEGM-based expansion medium or in differentiation medium (PNEUMACUL™-ALI); and during Day 1 to Day 3 of culture, the organoid was cultured in differentiation medium. FIG. 15B includes brightfield images of organoid integrity on Day 1 and Day 3 of culture.

FIGS. 16A and 16B show characterization of cellular composition in AOAOs differentiated in the presence and absence of IL-13. Specifically, immunofluorescence staining of MUC5AC, Ac-α-Tub, and FOXJ1 in AOAOs following 21 days culture in either differentiation medium (FIG. 16A) or in differentiation medium supplemented with IL-13 (5 ng/mL) (FIG. 16B) are shown.

FIGS. 17A-17E show assessment of epithelial polarity reversibility in organoids transitioned from suspension to MATRIGEL®-embedded culture. FIG. 17A is a diagram depicting human ABSC aggregates formed in ECM-free suspension culture for 1 day and then transferred into ECM-rich, MATRIGEL®-embedded culture and maintained for an additional 20 days. FIG. 17B includes immunofluorescence images of Day 21 organoids stained for FOXJ1, Ac-α-Tub, and ZO-1, as indicated. Scale bar, 25 μm. FIG. 17C is a graph plotting the percentage of Day 21 (D21) organoids with apical-out versus apical-in epithelial polarity, as indicated by apical Ac-α-Tub localization. FIGS. 17D and 17E are graphs plotting FOXJ1+ cell abundance (FIG. 17D) and percentage ciliation (FIG. 17E) in Day 21 (D21) organoids. Data represent means±s.d. from ≥15 organoids. ***, p<0.00¹.

FIGS. 18A and 18B show large organoid bodies in the organoid culture transferred to MATRIGEL® embedding following 1 day in suspension from dissociated hABSCs. Immunofluorescence staining of Ac-α-Tub (FIG. 18A) and FOXJ1 (FIG. 18B) in both large organoid bodies and regular-sized organoids on Day 21 of culture are shown.

FIGS. 19A-19F relate to enabling and quantifying AOAO rotation. FIG. 19A is a diagram depicting a method for enabling consistent AOAO rotational motion via matrix embedding. FIG. 19B is a diagram depicting a computational method for calculating an organoid's angular rotational motion: r0, the center of the organoid; rt, the position of the correspondence at time t; and rt+1, the position of the correspondence at time t+1. FIG. 19C includes a stepwise description of the computational framework used to calculate correspondence movement. FIG. 19D shows the rotational velocity φm/sec) and angular velocity (rad/sec) normalized by their mean values for a representative organoid. The X-axis is the position of correspondences along the X-axis of the ROI. The velocity of correspondences on the organoid, perpendicular to every position on the X-axis were projected onto the X-axis and averaged to obtain the rotational or angular velocity for that position. The velocities were then normalized by their mean values. The shaded regions represent the region of organoid which was used to calculate the rotational and angular velocity. FIG. 19E is a graph plotting the deviation in the angular and rotational velocity with respect to their mean values of 10 representative organoids from three independent replicates. The deviation was calculated by taking the mean squared difference between the velocity profile and its mean value. The deviation was then normalized by the mean value. FIG. 19F is a graph plotting the instantaneous angular velocity profile of 10 representative organoids from three independent replicates. The running mean (window=5) of instantaneous angular velocity for each organoid is shown as a solid line. ***, p<0.001.

FIGS. 20A and 20B are a pair of graphs plotting rotational (FIG. 20A) and angular (FIG. 20B) velocity profiles of AOAO rotation. The solid trace represents the average velocity of all correspondences on the organoid perpendicular to that position on the X-axis of the ROI. The dashed line represents the mean velocity profile.

FIGS. 21A-21H show the characterization of AOAO rotation and cilia motility in the presence of cilia beating inhibitors. FIG. 21A is a diagram showing AOAO rotational motion in response to EHNA or paclitaxel treatment. FIG. 21B is a diagram depicting computational methods used for calculating organoid angular velocity following 1 mM EHNA treatment for 2 hours. FIG. 21C is a graph plotting changes in organoid angular velocity following 2-hour treatment with EHNA at various concentrations. FIG. 21D shows CBF analyzed via conventional kymographs following 1 mM EHNA treatment for 2 hours, as well as graph plotting the CBF. FIG. 21E shows TEM imaging of ciliary ultrastructure with and without 24-hour paclitaxel (20 μM) treatment. Scale bar, 100 nm. Arrowheads indicate mislocated microtubules. FIG. 21F is a graph plotting data from a time-series analysis of paclitaxel's effect on AOAO angular velocity. FIG. 21G is a graph plotting changes in organoid angular velocity in the presence or absence of 20 μM paclitaxel treatment 24 hours. FIG. 21H shows CBF quantification via kymograph analysis in the presence or absence of 20 μM Paclitaxel for 24 hours. Data represent means±s.d. from three independent replicates with ≥10 organoids. **, p<0.01. * p<0.001.

FIGS. 22A-22H show modeling PCD-associated ciliary defects using AOAO rotation. FIG. 22A is a diagram depicting organoids engineered from hABSCs derived from PCD and healthy patients. FIG. 22B includes images of day-21 AOAOs (PCD and healthy) stained for Ac-α-Tub and ZO-1. Scale bar, 25 μm. FIG. 22C is a graph plotting percentage ciliation in PCD and healthy AOAOs. FIG. 22D includes TEM images of healthy and PCD AOAOs showing cilia ultrastructural defects in the PCD organoids (arrowheads). Scale bar, 100 nm. FIG. 22E is a diagram depicting the rotational motion of PCD and healthy AOAOs. FIG. 22F is a diagram showing computational methods used for calculating angular velocities of healthy and PCD AOAOs. FIG. 22G is a graph plotting rotation of MATRIGEL®-embedded, healthy and PCD AOAOs. FIG. 22H shows kymograph-based CBF quantification of healthy and PCD AOAOs. ***, p<0.001.

FIGS. 23A and 23B include a diagram (FIG. 23A) and images (FIG. 23B) showing diesel particulate matter (DPM) treatment of AOAO mature epithelium during Day 14 to Day 21. DPM, dispersed at 1 mg/mL in 0.5 wt % bovine serum albumin solution in ultrapure deionized water using a bath sonicator, was administered to AOAOs at 200 μg/mL from Day 14 to Day 21. Control AOAOs were exposed to 0.5 wt % bovine serum albumin solution.

FIGS. 24A and 24B show characterization of ciliation in mature AOAO epithelium after DPM treatment during day-14 to day-21. FIG. 24A includes immunofluorescence images of DPM treated and control AOAOs stained with cilia marker, Ac-α-Tub. Scale bar, 25 μm. FIG. 24B is a graph plotting percentage ciliation in AOAOs.

FIG. 25 includes immunofluorescence images showing characterization of DPM-treated mature AOAO epithelium for goblet cell hyperplasia and mucus hypersecretion. AOAOs were stained with the goblet cell marker, MUC5AC, following Day 14 to Day 21 culture with DPM. Scale bar, 25 μm.

FIGS. 26A-26F show characterization of DPM-induced respiratory injuries to the mature epithelium via three-dimensional (3D) AOAO rotation and two-dimensional (2D) AOAO locomotion. FIG. 26A is a diagram depicting and FIG. 26B is a graph plotting rotational analysis of MATRIGEL®-embedded AOAOs treated with DPM during day-14 to day-21 culture. FIG. 26C is a diagram depicting the locomotion of AOAOs on a 2D surface, driven by the beating of their exterior-facing cilia. The 2D locomotion was further characterized by a simultaneous revolutionary and self-rotary motion of the AOAO. FIG. 26D includes images showing the locomotion trajectories of DPM-treated AOAOs, and FIG. 26E is a graph plotting their calculated locomotion velocities. FIG. 26F is an image showing the 2D locomotion trajectories of a population of AOAOs.

FIGS. 27A-27C show the results of DPM treatment of the early-stage epithelium of Day 7 AOAOs for 14 days. FIG. 27A is a diagram depicting how AOAOs were treated with DPM during their differentiation into pseudostratified epithelium. Airway regeneration following DPM-induced airway injury was assessed by detecting the ciliated cell lineage specific marker Ac-α-Tub (FIG. 27B) and the goblet cell lineage specific markers MUC5AC (FIG. 27C). Scale bar, 25 μm.

### DETAILED DESCRIPTION

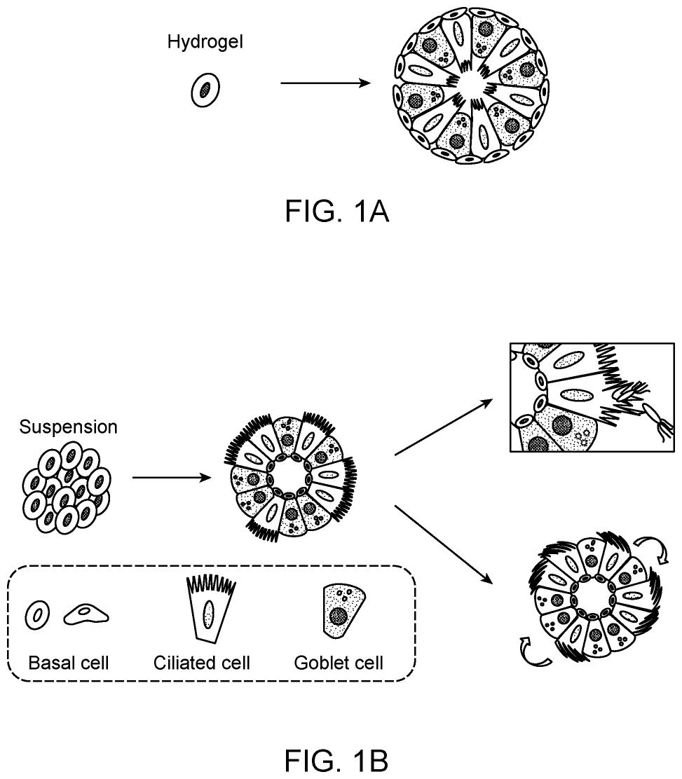

The human conducting airway is composed of polarized pseudostratified epithelium, with cilia movement and mucus secretion taking place on the apical side that faces the external environment and directly interacts with respiratory pathogens (“apical-out”). However, conventional airway organoids engineered from airway basal cells in extracellular matrix-embedded culture have an “apical-in” conformation (FIG. 1A) (Barkauskas et al., Development. 2017, 144:986-997; Dye et al., eLife. 2015, 4; McCauley et al., Cell Stem Cell. 2017, 20:844-857; Rock et al., Proc Natl Acad Sci USA. 2009, 106:12771-12775; and Sachs et al., EMBO J., 2019, 38), making the apical surface difficult to access and precluding effective investigation of airway pathology in a physiologically relevant manner.

This document provides methods and materials for engineering apical-out airway organoids by culturing basal cell clusters in suspension without any extracellular matrix support. The methods and materials provided herein can effectively be used to generate “apical-out” organoids having a polarity that is reversed as compared to “apical-in” organoids generated using previously described methods. In some cases, the methods and materials described herein can be applied to, for example, high-throughput investigation of respiratory infection and respiratory disorders, as well as analysis of candidate agents for treating respiratory infection and respiratory disorders, high-throughput analysis of cilia physiology and pathology, and real-time, non-invasive sampling and analysis of airway mucus production.

This document provides methods for making ciliated organoids having an apical-out polarity. In addition, this document provides organoids generated according to the methods described herein.

In some cases, the methods provided herein provide a simple yet robust approach for engineering apical-out human airway organoids (FIG. 1B) by culturing basal cell clusters in suspension without any extracellular matrix support. These apical-out airway organoids offer unique advantages over conventional apical-in organoids for investigating lung biology, respiratory infection, and other respiratory pathology.

In general, the methods for generating ciliated, apical-out organoids provided herein can include producing cellular aggregates or spheroids from any appropriate type of cells. For example, ciliated organoids can be generated from epithelial cells having an apical-basal polarity, such that they have an apical membrane on one side and a basal membrane on an opposite side. In an organ, the basal side of such a cell typically is anchored to other tissue via a basement membrane made up of a thin extracellular matrix (ECM) that contains a meshwork of proteins (e.g., laminins, collagen, and proteoglycans). Epithelial cells with an apical-basal polarity typically are free of attachment on the apical side, which can be exposed to the environment. For example, the apical side of airway cells is exposed to inhaled air, while the apical side of an intestinal cell is exposed to ingested food and liquid.

The apical membrane of the cells within the organoids produced and/or used according to the methods described herein also can be characterized by the presence of cilia. Cilia can be found on, for example, epithelial cells in the lungs and Fallopian tubes, as well as on ependymal cells that line brain vesicles. Cilia can move in a rhythmic waving manner to, for example, move debris and/or mucus along the cell surface. The methods for making organoids as described can include using ciliated cells, or precursor cells that will differentiate into ciliated cells. In some cases, the methods provided herein can include using airway basal cells, fallopian tube epithelial cells, middle ear epithelial cells, or brain ventricular epithelial cells. For example, airway basal cells such as normal human bronchial epithelial cells (NHBEs), airway bronchial stem cells (ABSCs), tracheal epithelial cells, nasal epithelial cells, or any combination thereof can be used to generate ciliated organoids. The cells can be obtained from any appropriate source. In some cases, from example, the cells can be primary cells obtained from a mammal (e.g., a human). In some cases, the cells can be derived from induced pluripotent stem cells. In some cases, the cells can be commercially obtained.

The methods provided herein for generating ciliated organoids can include suspending cells (e.g., airway basal cells) in an appropriate medium (e.g., a differentiation medium). Any suitable type of medium can be used. Without being bound by a particular mechanism, an air-liquid interface (ALI) medium (e.g., PNEUMACULT™-ALI medium) can be particularly useful. In some cases, the medium (e.g., the differentiation medium) can be substantially free from ECM and/or ECM components. A medium that is substantially free from ECM and/or ECM components is one that contains 5% or less by weight (e.g., 4% or less, 3% or less, 2% or less, 1% or less, by weight) of total ECM components. In some cases, a differentiation medium used in the methods provided herein does not contain any ECM or ECM components.

In some cases, a medium (e.g., a differentiation medium) used in the methods provided herein can contain one or more inhibitors of transforming growth factor β (TGFβ) and/or TGFβ kinase type 1 receptor. Suitable inhibitors of TGF and/or TGF kinase type 1 receptor include, without limitation, A8301, GW788388, RepSox, and SB 431542. In some cases, a differentiation medium used in the methods provided herein can contain one or more cytoskeletal structure modulators. For example, a differentiation medium can contain an inhibitor of Rho-associated protein kinase (ROCK), such as Y27632, SR 3677, thiazovivin, HA1100 hydrochloride, HA1077, or GSK-429286, an inhibitor of p21-activated kinase (PAK), such as IPA3, and/or an inhibitor of myosin II, such as blebbistatin. In some cases, a differentiation medium used in the methods provided herein can contain one or more inhibitors of BMP4/SMAD signaling. For example, a differentiation medium can contain DMH-1. In some cases, a differentiation medium used in a method provided herein can contain one or more activators of the WNT pathway. For example, a differentiation medium can contain CHIR99021. It is to be noted that a differentiation medium used in the methods provided herein can contain any combination of the aforementioned components.

Once a population of cells (e.g., airway basal cells) have been suspended in a differentiation medium, they can be cultured under conditions such that the cells aggregate and differentiate. A portion of the suspended and aggregated cells can be transferred into an appropriate receptacle for differentiation and organoid generation. Any appropriate number of cells can be transferred. For example, about 20 to about 10,000 cells (e.g., about 20 to about 50, about 50 to about 100, about 100 to about 200, about 200 to about 300, about 300 to about 500, about 500 to about 1000, about 1000 to about 2500, about 2500 to about 5000, about 5000 to about 10,000, about 20 to about 100, about 50 to about 500, about 100 to about 1000, about 20, about 50, about 200, about 500, or about 1000 cells) can be placed in a receptacle and cultured to form organoids.

Any suitable type of receptacle can be used. In some cases, for example, a microplate having multiple wells can be used, and aliquots cells can be placed into one or more wells of the microplate. The microplate wells can be of any size, and can have, for example, 12 wells, 24 wells, 48 wells, 96 wells, 192 wells, 384 wells, or 1536 wells. In some cases, the wells of the microplate can have a cell-repellent coating such that the cells do adhere to the plate. For example, in some cases, a cell-repellent 96-well microplate can be used. Moreover, the wells can have any appropriate geometry. For example, the wells can have a flat bottom, a U-shape with a rounded bottom surface, or a V-shape with a sharp point at the very bottom.

The receptacle containing the differentiated cells can then be maintained for an appropriate length of time under conditions such that ciliated organoids having apical-out polarity can form. For example, a microplate containing differentiated cells (e.g., human airway basal cells) can be maintained for about 7 to 42 days (e.g., about 7 to 14 days, about 14 to 21 days, about 21 to 28 days, about 28 to 35 days, about 35 to 42 days, about 7 to 21 days, about 14 to 28 days, or about 21 to 35 days) at about 37° C. with about 5% CO₂. In some cases, the medium in which an organoid is cultured can be replaced (either fully or partially) periodically, such as on a daily basis, every other day, or every third day. Once spheroid has differentiated into an organoid, it can be maintained in the same medium, or it can be transferred to a different medium (e.g., basal medium, such as Dulbecco's Modified Eagle Medium/Nutrient Mixture F-12) or to a three-dimensional hydrogel material, such as MATRIGEL© (a solubilized basement membrane matrix secreted by Engelbreth-Holm-Swarm mouse sarcoma cells; CORNING®/Thermo Fisher Scientific, Waltham, MA).

In some cases, the formation of spheroids and then organoids can be monitored by visual inspection of the cells in the receptacle (e.g., with a microscope), such that their shape, and size can be assessed, and the presence and movement of cilia on organoids can be noted. Any other suitable method can be used to assess the organoids. For example, immunofluorescence techniques can be used to assess the expression of certain markers.

As described in the Examples herein, for example, organoids can be assessed for expression of an airway basal epithelial cell marker (e.g., p63), a ciliated cell nuclear marker (e.g., FOXJ1), a cilia marker (e.g., acetylated alpha tubulin), a goblet cell marker (e.g., MIUC5AC), and/or Zonula occludens-1 (ZO-1, a classical scaffold protein with roles in maintaining cell-cell adhesions in stable tissues) to determine their quality, polarity, and ciliation status.

In some cases, organoids generated as described herein can contain one or more non-ciliated cell types in addition to the ciliated epithelial cells. For example, an organoid can further contain stromal cells (e.g., fibroblasts, pericytes, mesenchymal stem cells, adipose stem cells, myofibroblasts, and/or lipofibroblast), vascular endothelial cells, immune cells (e.g., lymphocytes, neutrophils, eosinophils, basophils, mast cells, monocytes, macrophages, dendritic cells, and/or natural killer cells), or any combination thereof. When such non-ciliated cell types are included in an organoid, they can make up any appropriate percentage of the cells in the organoid. For example, an organoid can contain from about 1% to about 50% non-ciliated cells (e.g., about 1 to about 10%, about 10 to about 20%, about 20 to about 30%, about 30 to about 40%, or about 40 to about 50%) non-ciliated cells, and about 50 to about 99% ciliated cells (e.g., about 50 to about 60%, about 60 to about 70%, about 70 to about 80%, about 80 to about 90%, or about 90 to about 99% ciliated cells).

The ciliated organoids prepared according to the methods described herein can exhibit particular physical characteristics. For example, an organoid can exhibit coordinated ciliary beating. The coordinated beating for the cilia on a nanoscale can be transformed into microscale organoid motility, such that the cilia on the organoid move in a coordinated fashion and confer movement to the organoid. In some cases, coordinated ciliary beating can confer 3D rotational movement to an organoid. In some cases, coordinated ciliary beating can confer 2D motility (locomotion) to an organoid, such that it moves in a particular path. For example, the coordinated beating of cilia on an organoid can confer motility along a repeating, spiral pathway. Methods for assessing characteristics such as ciliary beating, 3D rotation, and 2D locomotion, as well as goblet cell hyperplasia and mucus secretion, are described in the Examples herein.

Given such reproducible physical characteristics, this document also provides methods for using ciliated organoids (e.g., AOAOs) generated according to the methods described herein. In general, methods for using the ciliated organoids can include contacting one or more ciliated organoids with an agent that may have an effect on at least one physical characteristic of the organoid, such as its ciliary movement (e.g., the coordinated beating of the cilia on the organoid's external surface), the 2D motility of the organoid, or the 3D rotation of the organoid. An “agent” can be something (e.g., a molecule or a microbe) that can physically contact an organoid, or can be something that does not physically contact the organoid but can still have an effect on the organoid. Agents that can be evaluated include, without limitation, potential pathogens (e.g., bacteria and viruses), pollutants (e.g., particulates, carbon monoxide, nitrogen oxide, sulfur oxide, ozone, and secondary organic aerosols), biological or chemical agents (e.g., cytokines, such as inflammatory and/or pro-fibrotic cytokines (e.g., interleukin-1p, interleukin-6, tumor necrosis factor-α, and granulocyte macrophage colony-stimulating factor), or cytotoxic biological or chemical agents, such as chemotherapeutics) and therapeutic agents or candidates (e.g., small molecules, antibodies, polypeptides, and nucleic acids). Other agents that can be evaluated include, without limitation, environmental conditions such as temperature and humidity, or other potential effectors such as irradiation (e.g., x-rays, gamma rays, proton radiation, alpha radiation, beta radiation, or neutron radiation).

By measuring or otherwise evaluating one or more organoid characteristics before exposure to an agent and also during and/or after exposure to the agent, the effect of the agent (if any) on the organoid can be determined. Since the organoids described herein are stable and can be non-invasively contacted with a test agent, they can be treated with an agent for a suitable length of time, and can be evaluated days or even weeks after initiation or cessation of treatment. For example, an organoid can be treated with an agent for 30 seconds to several weeks (e.g., 30 to 60 seconds, 1 to 60 minutes, 1 to 4 hours, 4 to 8 hours, 8 to 12 hours, 12 to 24 hours, 1 to 2 days, 2 to 3 days, 3 to 7 days, 1 to 2 weeks, or 2 to 3 weeks). The organoid can be assessed any suitable length of time after treatment has commenced and/or, in some cases, after treatment has ended. For example, one or more organoid characteristics can be evaluated an hour to 28 days (e.g., 1 to 2 hours, 2 to 4 hours, 4 to 6 hours, 6 to 12 hours, 12 to 24 hours, 1 to 2 days, 2 to 4 days, 4 to 7 days, 7 to 14 days, 14 to 21 days, or 21 to 28 days) after treatment has commenced or after treatment has ended.

Several representative examples of applications that can benefit from the use of ciliated organoids are described below.

High-throughput investigation of respiratory infection. In native human lung, the apical airway surface is exposed directly to the external environment and therefore is the main interface for host interaction with respiratory pathogens, such as bacteria and viruses. In conventional apical-in organoids, the apical surface of airway epithelium is hidden inside the organoids, and can only be accessed by microinjection, which is not only time-consuming but also invasive, with high risk of damaging the organoids (Porotto et al., mBio. 2019, 10:e00723-19). The apical-out conformation enabled by the methods provided herein allows for convenient introduction of respiratory pathogens directly to the apical surface of the airway organoids, which can closely recapitulate the native interactions between airway epithelium and respiratory pathogens (FIG. 1C). Thus, these apical-out organoids enable high-throughput investigation of host-pathogen interaction in the respiratory system and expedite therapeutic development.

High-throughput analysis of cilia physiology and pathology. Cilia in the human airway are located at the apical surface of ciliated cells. Cilia beat in a coordinated fashion, at a synchronized frequency, which generates a wave at the airway luminal surface and propel the overlying mucus in the cephalad direction (Bustamante-Marin and Ostrowski, Cold Spring Harbor Perspec Biol. 2017, 9:a028241). Cilia beating defects are observed in genetic disorders such as primary ciliary dyskinesia (PCD) and cystic fibrosis (CF), and in acquired disorders associated with cigarette smoking, environmental pollution, chronic obstructive pulmonary disease, bronchiectasis, asthma, acute and chronic infection, interstitial lung disease, lung transplant, bone marrow transplant, mechanical ventilation, sepsis, and lung cancer (Tilley et al, Ann Rev Physiol. 2015, 77:379-406). Therefore, efficient characterization of cilia beating phenotype is critical for proper clinical diagnosis of cilia disorders and for therapeutic development. However, due to the small size of cilia, which are about 6 to 7 μm in length and about 0.2 to 0.3 m in diameter, and their high beating frequency (about 12-16 Hz) (Yaghi and Dolovich, Cells. 2016, 5:40; and Satir and Christensen, Ann Rev Physiol. 2007, 69:377-400), characterization of cilia beating phenotype generally requires the use of specialized high-speed video cameras at high magnification (Yaghi and Dolovich, supra; Schipor et al., Am J Rhinol. 2006, 20:124-127; Dimova et al., J Pharm Pharmacol. 2005, 57:521-526; and Quinn et al., Science Translational Medicine. 2015, 7:299ra124-299ra124). This process is cumbersome, costly, and challenging to scale up to high-throughput format. Enabled by the described apical-out airway organoids, cilia beating on the outer organoid surface leads to rotary motion of the organoid with a frequency of 0.5-2 Hz at the scale of 50-200 μm, which can be readily documented using conventional microscopes (FIG. 1D). This technology further enables simultaneous imaging and analysis of the cilia beating phenotype of a large number of organoids in a high-throughput manner.

Real-time, non-invasive sampling and analysis of airway mucus production. In the native human airway, mucus is primarily produced by goblet cells into the apical surface of airway epithelium. Mucus secretion can be stimulated by bacteria, particles and chemical irritants. Mucus hypersecretion represents a major clinical and pathological feature in cystic fibrosis, bronchiectasis, chronic obstructive pulmonary disease, and asthma (Shale and Ionescu, Eur Resp J. 2004, 23:797-798). Characterization of the quantity, composition and dynamics of airway mucus secretion in airway disease models is essential for mechanistic investigation as well as therapeutic development. However, in conventional apical-in organoids, mucus is secreted into the completely enclosed organoid lumen, and is therefore difficult to sampled without compromising the organoid integrity, making this an end-point assay. Enabled by the described apical-out airway organoids, mucus is secreted to the exterior organoid surface, allowing real-time, non-invasive mucus sampling and analysis.

The invention will be further described in the following examples, which do not limit the scope of the invention described in the claims.

### EXAMPLES

### Example 1—Materials for Airway Basal Cell Culture and Differentiation

Materials used for airway basal cell culture and expansion/aggregation included:
NHBE cells without retinoic acid (Lonza, Basel, Switzerland; CC-2541).
804G rat bladder epithelial cells
Bronchial Epithelial Cell Growth Basal Medium (BEBM™; Lonza, Basel, Switzerland; CC-3171)
BEGM™ SINGLEQUOTS™ supplements and growth factors (Lonza; CC-4175).
A8301 inhibitor of transforming growth factor β kinase type 1 receptor (Sigma-Aldrich, St. Louis, MO; SML0788-5MG)
Y27632 inhibitor of ROCKs (Cayman Chemical, Ann Arbor, MI; 129830-38-2)
DMH-1 inhibitor of BMP4/SMAD signaling (Tocris Bioscience, Minneapolis, MN; 4126)
CHIR99021 activator of the WNT pathway (Reprocell, Yokohama, Japan; 04000402)
RPMI 1640 with L-glutamine (CORNING®/Thermo Fisher Scientific, Waltham, MA; 10-040-CV)
HYCLONE™ FETALCLONE™ I Serum (GE Healthcare, Chicago, IL; SH30080.03)
Penicillin-Streptomycin (Lonza; 17-602E)
Dulbecco's Phosphate-Buffered Saline (DPBS), 1× without calcium and magnesium (CORNING®/Thermo Fisher Scientific; 21-031-CV)
CRYOSTOR® cs2 freeze media (BioLife Solutions, Bothell, WA; 202102)
0.25% Trypsin-EDTA (GIBCO™/Thermo Fisher Scientific; 25200056)
75 cm² cell culture flasks (Greiner Bio-One, Monroe, NC; 658175)
175 cm² cell culture flask (Greiner Bio-One; 660175)
25 mm, 0.45 μm pore size syringe filters (GE Healthcare; 67/802,504)
Sterile 30 mL luer-lok tip syringes (Becton Dickinson, Franklin Lakes, NJ; 302832)
15 mL centrifuge tubes (Thermo Fisher Scientific; 14-955-237)

Materials used for mucociliary differentiation of airway basal cells with apical-out polarity and embedding to induce rotary motion included:
PNUEMACULT™-ALI Basal Medium (STEMCELL™ Technologies, Vancouver, BC; 05002)
PNEUMACULT™-ALI 10× Supplement (STEMCELL™ Technologies; 05003)
PNEUMACULT™-ALI Maintenance Supplement (STEMCELL™ Technologies; 05006)
Heparin solution (STEMCELL™ Technologies; 07980)
Hydrocortisone stock solution (STEMCELL™ Technologies; 07925)
96-well cell-repellent microplate with lid (Greiner Bio-One; 655970)
25 mm, 0.45 μm pore size syringe filters (GE Healthcare; 67/802,504)
Sterile 30 mL luer-lok tip syringes (Becton Dickinson; 302832)
Growth factor reduced MATRIGEL® (CORNING®; CB 40230)

### Example 2—Culture and Differentiation of Airway Basal Stem Cells to Produce AOAOs

To prepare complete airway basal cell culture medium, the contents of the BEGM™ SINGLEQUOTS® supplements and growth factors were transferred into BEBM®, and the prepared medium was then supplemented with 1 μM A8301, 5 μM Y27632, 0.2 μM DMH-1, and 0.5 μM CHIR99021.

To prepare 804G conditioned medium, complete RPMI culture medium was prepared by combining RPMI 1640 with L-glutamine with 10% HYCLONE™ FETALCLONE™ I Serum and 1% Penicillin-Streptomycin. A vial of 804G rat bladder cells was thawed and placed into a 175-cm² cell culture flask with complete RPMI culture medium. The next day, the medium was aspirated and fresh medium was added. The cells were cultured until they reached about 90% confluence, while changing the medium every 2 to 3 days. Once the cells reached confluency, the medium was aspirated and 50 mL of fresh complete RPMI medium was added. The medium was then collected, and 50 mL of fresh complete RPMI medium was added every other day, up to four times. All collected media were combined and filtered through 0.45 m syringe filters.

For culture of airway basal stem cells, three 75-cm² cell culture flasks were pre-coated by adding 10 mL of prepared 804G-conditioned medium and incubating at 37° C. overnight. The 804G-conditioned medium was aspirated and the flasks were rinsed once with DPBS. Warm complete airway basal cell culture medium was added to the pre-coated 75-cm² cell culture flasks. A vial of NHBEs (˜800,000 cells) was thawed and added to the three flasks. The cell seeding density of each flask was about 3500 cell/cm². The medium was aspired the next day and fresh complete airway basal cell culture medium was added. The medium was changed every other day.

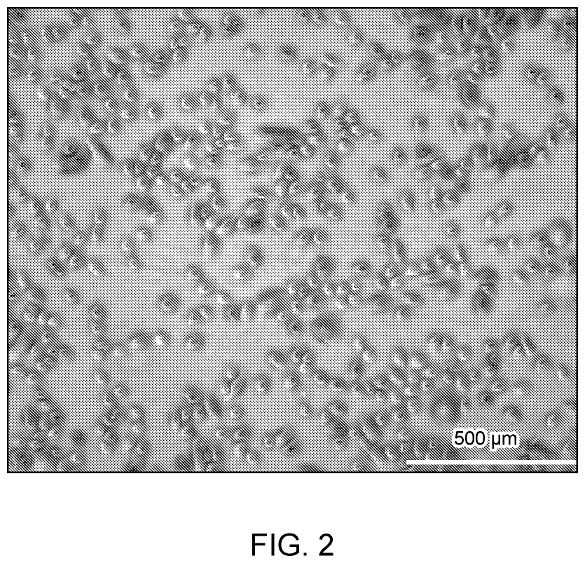

For expansion of airway basal stem cells (FIG. 2), once the cells reached about 75% confluence, the medium was aspired and the flasks were rinsed once with DPBS. Trypsin-EDTA (0.25%) was added and the flasks were incubated at 37° C. After all the cells had lifted, complete RPMI medium was added to neutralize the trypsin. The cell suspension was transferred into a 15 mL centrifuge tube, which was then centrifuged at 220×g for 5 minutes at room temperature. The supernatant was removed. For cryopreservation, the cell pellet was resuspended in CRYOSTOR® cs2 freeze media to a final cell density of 500,000 cells/mL. One (1) mL of cell suspension was transferred into each cryovial and frozen. For expansion, the cell pellet was resuspended in complete airway basal cell culture medium and seeded in an 804G conditioned medium-coated flask at a density of about 3500 cells/mL.

Before differentiation of airway basal stem cells into apical-out organoids, PNEUMACULT™-ALI Complete Base Medium was prepared by adding 50 mL of PNEUMACULT™-ALI 10× Supplement to 450 mL PNEUMACULT™-ALI Basal Medium according to the manufacturer's instructions. Differentiation medium was prepared by combining 9.83 mL PNEUMACULT™-ALI Complete Base Medium, 100 μL PNEUMACULT™-ALI Maintenance Supplement, 20 μL heparin solution, and 50 μL Hydrocortisone Stock Solution.

Mucociliary differentiation of NHBEs with apical-out polarity was then carried out. Once the cells reached about 75% confluency, the medium was aspirated and cells were rinsed once with DPBS. Trypsin-EDTA (0.25%) was added and incubated at 37° C. After all the cells had lifted, complete RPMI medium was added to neutralize the trypsin. The cell suspension was transferred into a 15 mL centrifuge tube, which was then centrifuged at 220×g for 5 minutes at room temperature. The cell pellet was resuspended to a cell density of 5000 cells/mL in differentiation medium containing 10 μM of Y27632. Aliquots (100 μL) of the cell suspension were then transferred to each well of a 96-well cell-repellent microplate. 50 μL of the media were pipetted out from each well and replaced with fresh differentiation medium every other day.

The apical-out organoids were then embedded in MATRIGEL®. Once the apical-out organoids reached day 21, they were collected from the 96-well cell-repellent microplates (using a wide-bore pipet tip) and placed into a 1.5 mL Eppendorf tube. Organoids were allowed to settle at the bottom of the tube for 10 minutes. The medium was removed until 100 μL was left in the 1.5 mL Eppendorf tube. The organoids were then transferred into a culture well of a flat-bottom cell culture 96-well plate. Medium was removed until 60 μL was left in the well. Cold growth factor reduced MATRIGEL® (40 μL) was added to the well and pipetted up and down several times to mix. The 96-well plate was left on a hot plate set to 37° C. for 10 minutes, after which 100 μL of medium was added and the plate was cultured in a cell culture incubator.

### Example 3—Characterization of Apical-Out Airway Organoids

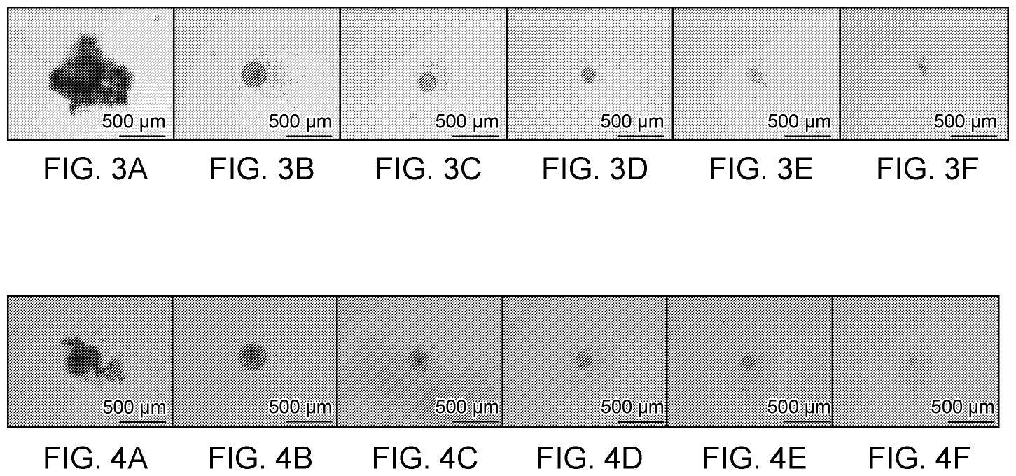

Apical-out organoid formation was examined with different starting NHBE cell numbers (1000, 500, 200, 100, 50, and 20 cells per well) on a 96-well U-bottom cell repellent surface. Studies also were conducted to test whether the addition of 10 μM Y27632 into the differentiation medium for initial seeding improved cell viability and spheroid formation (FIGS. 3A-3F and 4A-4F). Although there were no significant differences between spheroids formed with (FIGS. 4A-4F) and without (FIGS. 3A-3F) Y27632, differentiation medium with Y27632 was chosen for cell seeding. In addition, since the highest cell density of 1000 cells per well did not support favorable spheroid formation in these studies (FIGS. 3A and 4A), a density of 500 cells per well (FIGS. 3B and 4B) was selected for further studies. It is noted that in other experiments, however, a density of 1000 cells per well did support favorable spheroid formation.

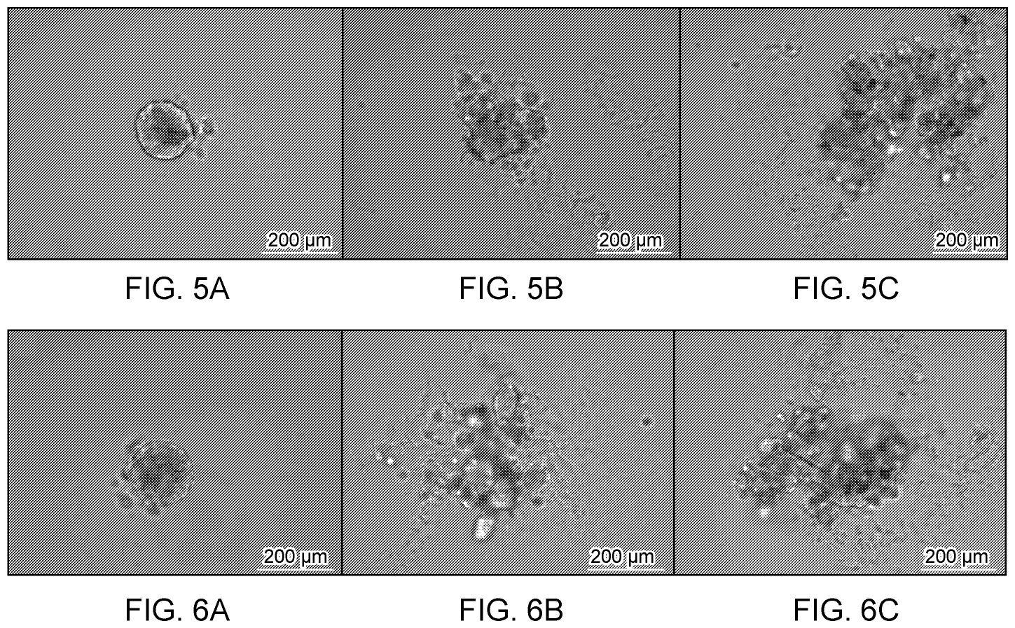

The importance of cell medium and agitation of the plate for spheroid formation on the U-bottom cell repellent surface was investigated. In these studies, as shown in FIGS. 5A-5C and 6A-6C, the NHBEs failed to form spheroids in BEGM™ or BEGM™ with added small molecule factors, even with agitating support of the well plate at 125 RPM.

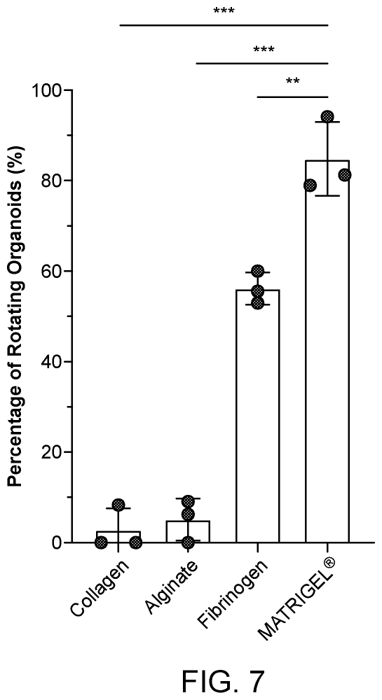

Within 14 days of culture, cilia beating was observed on the exterior surface of the organoids (apical-out organoids); the beating became more prominent over time. Further, cilia beating powered the apical-out organoids to rotate on their own when embedded in an appropriate soft supporting matrix. Differentiated organoids were embedded in various matrices, including collagen, alginate, fibrinogen, and MATRIGEL®, to identify the best matrix for support the organoids' rotary motion. In these studies, the use of MATRIGEL® resulted in significantly better organoid rotation than the other matrices tested (FIG. 7).

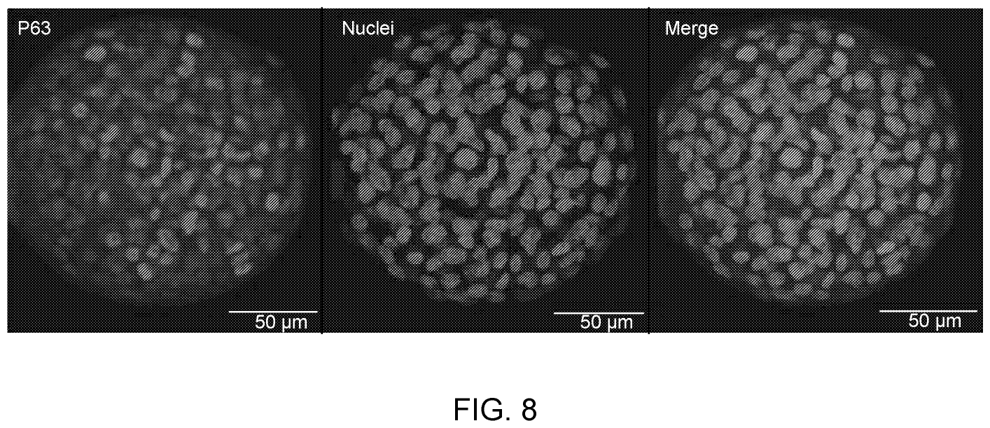

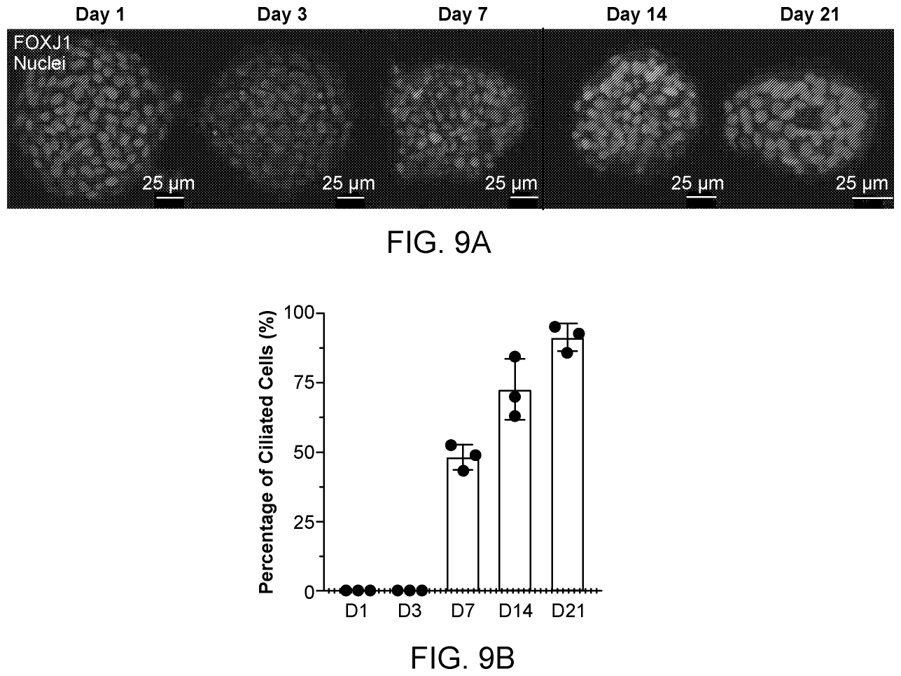

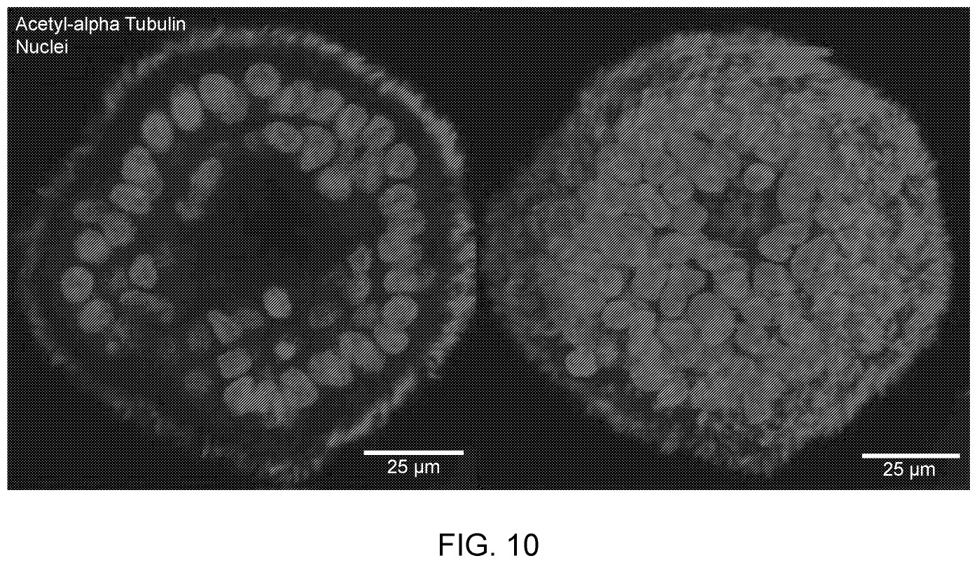

The cellular composition of apical-out organoids was evaluated at different stages via wholemount immunofluorescence staining. At the early stage of Day 1, organoids consisted mostly of epithelial basal cells (FIG. 8). By Day 7, the organoids expressed ciliated cells, and by Day 21, almost all (more than 90%) of the epithelial basal cells had differentiated into ciliated cells (FIGS. 9A and 9B). Mature differentiated organoids also showed strong evidence of cilia located on the apical surface on Day 21 when stained for the cilia marker, acetylated alpha-tubulin (FIG. 10).

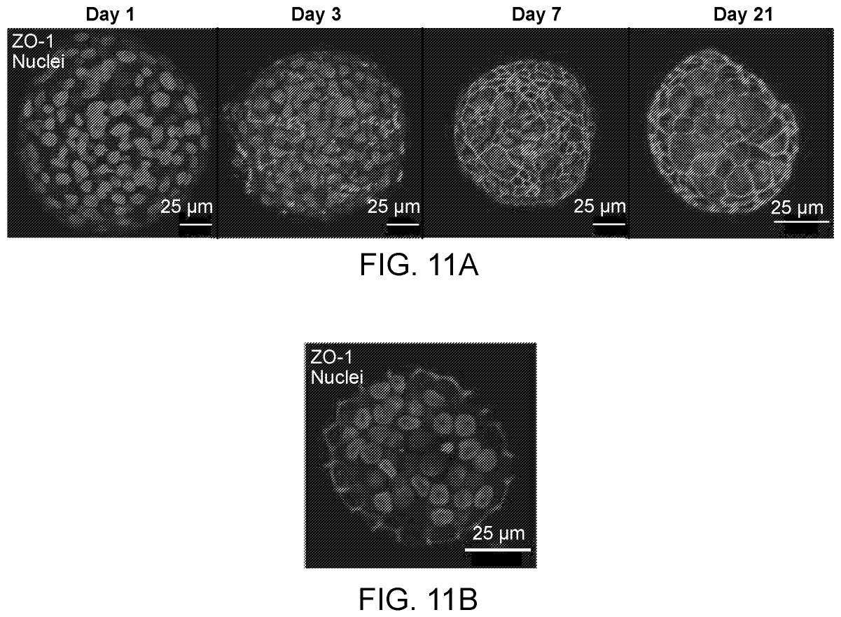

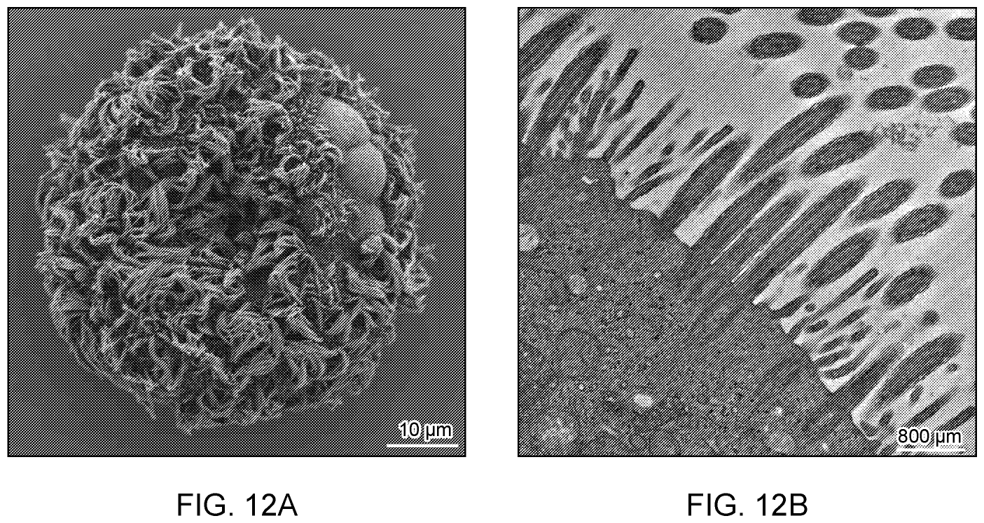

The polarity reversal and apical out phenotype of the organoids was investigated using the epithelial tight junction protein, zonula occludens-1 (ZO-1). The organoids began to express ZO-1 in random patterns on Day 3. By Day 7, ZO-1 expression had become more defined (FIG. 11A). The ZO-1 expression migrated towards the organoid's apical surface by Day 21, indicating polarity reversal from apical-in to apical-out (FIG. 11B). Scanning electron microscopy (SEM) and transmission electron microscopy (TEM) images also were used to further verify the apical-out phenotype of the organoids (FIGS. 12A and 12B).

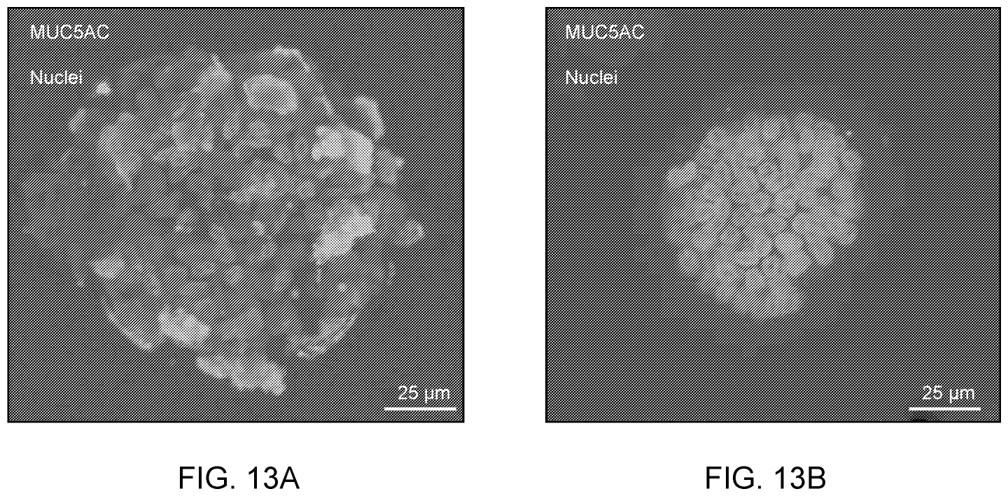

The inflammatory responses of the apical-out organoids was assessed by treating the organoids with the pro-inflammatory cytokine interleukin-13 (IL-13). These studies revealed that organoids treated with IL-13, even at concentration as low as 1 ng/mL, exhibited goblet cells (FIG. 13A). In comparison, non-treated organoids had no MUC5AC expression, indicating the absence of goblet cells and no proinflammatory response (FIG. 13B).

### Example 4—Materials and Methods for Examples 5-9

Reagents and Equipment: The reagents and equipment used in the studies described in Examples 5 to 9 are listed in TABLES 1 to 4.

TABLE 1
Reagents

| Name | Source | Catalog No. |
|---|---|---|
| A8301 (inhibitor of transforming growth | Sigma-Aldrich | SML0788-5MG |
| factor β kinase type 1 receptor) | | |
| BEBM ™ | Lonza | CC-3171 |
| BEGM ™ SINGLEQUOTS ™ supplements | Lonza | CC-4175 |
| and growth factors | | |
| Bovine Collagen, Type I | Advanced | 5225 |
| | BioMatrix | |
| CHIR99021 (activator of WNT pathway) | Reprocell | 04000402 |
| DPBS, 1X without calcium and magnesium | CORNING ® | 21-031-CV |
| DMH-1 (inhibitor of BMP4/SMAD signaling) | Tocris Bioscience | 4126 |
| EHNA, hydrochloride (inhibitor of dynein) | Millipore Sigma | 32-463-010MG |
| Growth factor reduced MATRIGEL ® | CORNING ® | CB 40230 |
| Heparin solution | STEMCELL ™ | 07980 |
| | Technologies | |
| HYCLONE ™ FETALCLONE ™ I Serum | GE Healthcare | SH30080.03 |
| (fetal bovine serum alternative) | | |
| Hydrocortisone stock solution | STEMCELL ™ | 07925 |
| | Technologies | |
| NHBE cells without retinoic acid | Lonza | CC-2541 |
| Paclitaxel (inhibitor of microtubules) | Cayman Chemicals | 10461 |
| PNEUMACULT ™-ALI Medium | STEMCELL ™ | 05001 |
| | Technologies | |
| Penicillin-Streptomycin | Lonza Walkersville | 17-602E |
| | Inc. | |
| RPMI 1640 with L-glutamine | CORNING ® | 10-040-CV |
| Y27632 (Inhibitor of ROCKs) | Cayman Chemicals | 129830-38-2 |
| 0.25% Trypsin-EDTA | GIBCO ™ | 25200056 |
| 96-well cell-repellent microplate with lid | GreinerBio-One | 655970 |

TABLE 2
Cells

| Name | Source | Catalog No. |
|---|---|---|
| Bronchus-derived human | Lonza | CC-2541 |
| airway basal stem cells | | |
| Airway basal stem cells derived | Washington | |
| from de-identified tissues of | University | |
| healthy lung donor or donor with | | |
| primary ciliary dyskinesia | | |
| (carrying mutations in CCDC39 | | |
| (c.830_831delCA (p.Thr277Argfs*3) and | | |
| c.1871_1872delTA (p.Ile624Lysfs*3)) | | |

TABLE 3
Antibodies

| Name | Source | Catalog No. | Dilution |
|---|---|---|---|
| Mouse Anti-ZO-1 | Invitrogen | 33-9100 | 1:300 |
| Mouse Anti-acetylated- | Sigma-Aldrich | T6793 | 1:200 |
| α-Tubulin | | | |
| Mouse Anti- MUC5AC | Thermo Fisher | MA5-12178 | 1:200 |
| | Scientific | | |
| Mouse Anti-TRP63 | Biocare | cm163a | 1:200 |
| | Medical | | |
| Mouse Anti-FOXJ1 | Thermo Fisher | 14-9965-82 | 1:100 |
| | Scientific | | |
| Donkey anti-Mouse Secondary | Thermo Fisher | A-21202 | 1:1000 |
| Antibody, Alexa Fluor 488 | Scientific | | |
| Donkey anti-Mouse Secondary | Thermo Fisher | A-31571 | 1:1000 |
| Antibody, Alexa Fluor 647 | Scientific | | |

TABLE 4
Equipment

| Name | Source | Catalog No. |
|---|---|---|
| EVOS FL Auto 2 | Thermo Fisher | AMAFD2000 |
| Imaging System | Scientific | |
| Confocal Microscope | Zeiss | LSM 700 |
| Confocal Microscope | Zeiss | AxioObserver Z1 |
| High-speed sCMOS camera | PCO | pco.edge 5.5 |

Culture of airway basal cells: Bronchus-derived human airway basal stem cells (hABSCs) were purchased from Lonza. Additional hABSCs were obtained from surgical excess of de-identified tissues of healthy lung donors or donors carrying mutations in the CCDC39 gene (c.830_831delCA(p.Thr277Argfs*3) and c.1871_1872delTA (p.Ile624Lysfs*3)). The hABSCs were cultured in 804G-conditioned medium coated culture vessels in BEGMm supplemented with 1 μM A8301, 5 μM Y27632, 0.2 μM 359 DMH-1, and 0.5 μM CHIR99021 at 37° C. with 5% C02. (Levardon et al., Bio Protoc 8, doi:10.21769/BioProtoc.2877, 2018).

Differentiation of airway basal cells into apical-out airway organoids: Human ABSCs (P2) were trypsinized and resuspended (5000 cells/ml) in differentiation medium (PNEUMACUL™-ALI Medium) supplemented with 10 μM Y27632. 100 μL of resuspended hABSCs were placed per well in a 96-well cell-repellent microplate (GreinerBio-One, 655970). The cultures were maintained at 37° C. with 5% CO₂ for 21-28 days. To assess how IL-13 modulated organoid maturation, IL-13 (5 ng/mL) was supplemented to the differentiation medium during the entire differentiation period.

To assess organoid polarity in response to two-phase (ECM-deprived and then ECM-supported) culture, day-1 organoids formed in a 96-well cell-repellent microplate were collected, resuspended in 40% (vol/vol) growth factor reduced (GFR) MATRIGEL® and added to a new well plate that had been pre-coated with 40% GFR-MATRIGEL®. The culture was then continued for an additional 20 days in differentiation medium.

Immunofluorescence staining: Airway organoids were collected from the 96-well cell-repellent microplate and fixed with 4% paraformaldehyde (PFA) for 1 hour at 4° C. The fixed organoids were washed with PBS with 0.1% Tween-20 and permeabilized with 1% Triton-X for 1 hour before incubating with primary antibodies diluted in 1% bovine serum albumin (BSA) overnight. Next, the organoids were washed with PBS 377 with 0.1% Tween20 and incubated with secondary antibodies. The nuclei were stained with 4′,6-diamidino-2-phenylindole (DAPI) before capturing z-stacks of stained organoids on a Zeiss LSM 700 laser scanning confocal microscope.

Scanning electron microscopy: The AOAOs were fixed with 2.5% glutaraldehyde in 0.01 M PBS (pH 7.4) for 1 hour at room temperature. The organoids were washed 3 times in 0.01 M PBS and then post-fixed with aqueous 1% osmium tetroxide for 1 hour at 4° C. Next, the organoids were rinsed 3 times in 0.01 M PBS before dehydrating in a graded series of 30%, 50%, 70%, and 90% ethanol, followed by 3 changes in 100% ethanol. The organoids were further dehydrated in hexamethyldisilazane for 15 minutes and allowed to air dry. The fixed and dehydrated organoids were mounted on studs and sputter-coated with 5 nm gold-palladium alloy prior to imaging with JEOL JSM 7800.

Transmission electron microscopy: The AOAOs were rinsed in 0.01 M PBS and fixed with 2.5% glutaraldehyde in 0.01M PBS (pH 7.4) for 1 hour at room temperature. The organoids were washed 3 times in 0.01M PBS and then post fixed with aqueous 1% osmium tetroxide containing 1% potassium ferricyanide for 1 hour at 4° C. Next, the organoids were rinsed 3 times in 0.01 M PBS before dehydrating in a graded series of 394 30%, 50%, 70%, and 90% ethanol, followed by 3 changes in 100% ethanol. The organoids were washed in Polybed 812 epoxy resin for 3 times for 1 hour each before polymerizing at 37° C. overnight and then for additional 48 hours at 60° C. Finally, the prepared organoid samples were sectioned at 60 nm, placed on copper grids, and imaged with JEM 1400 Flash TEM.

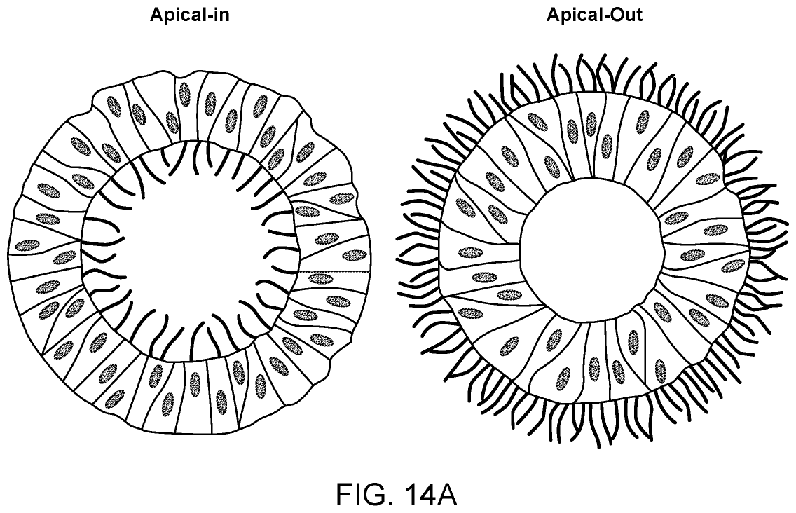

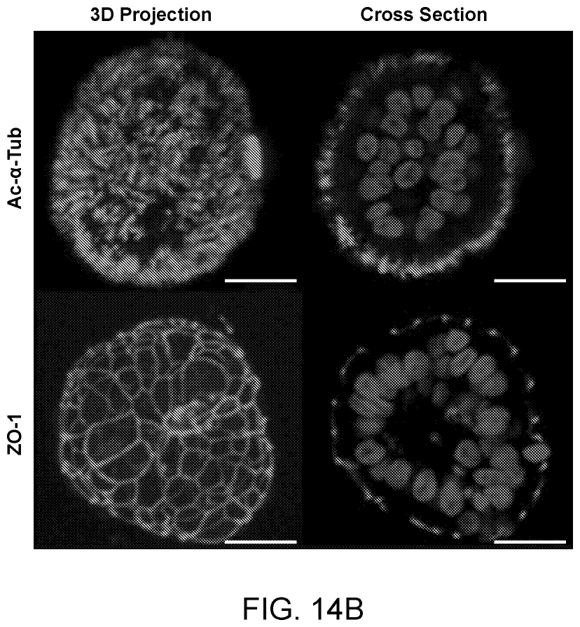

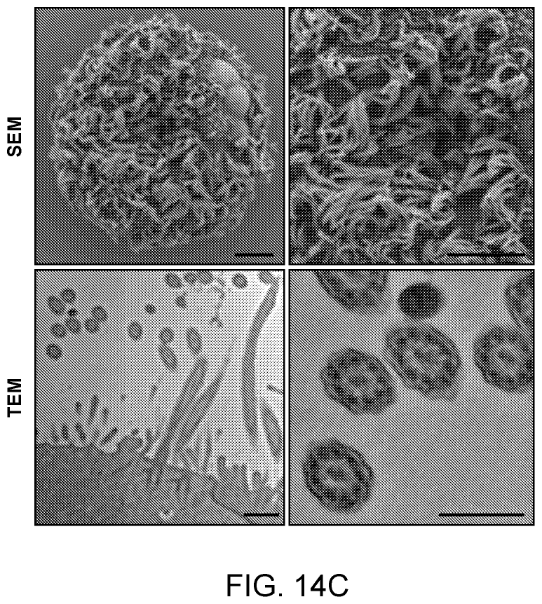

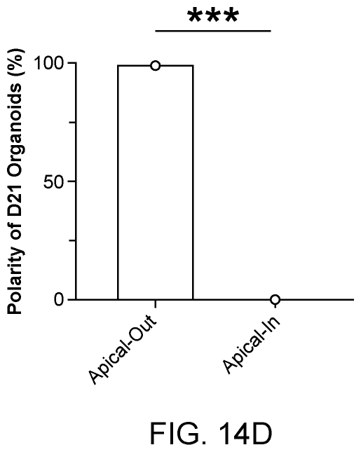

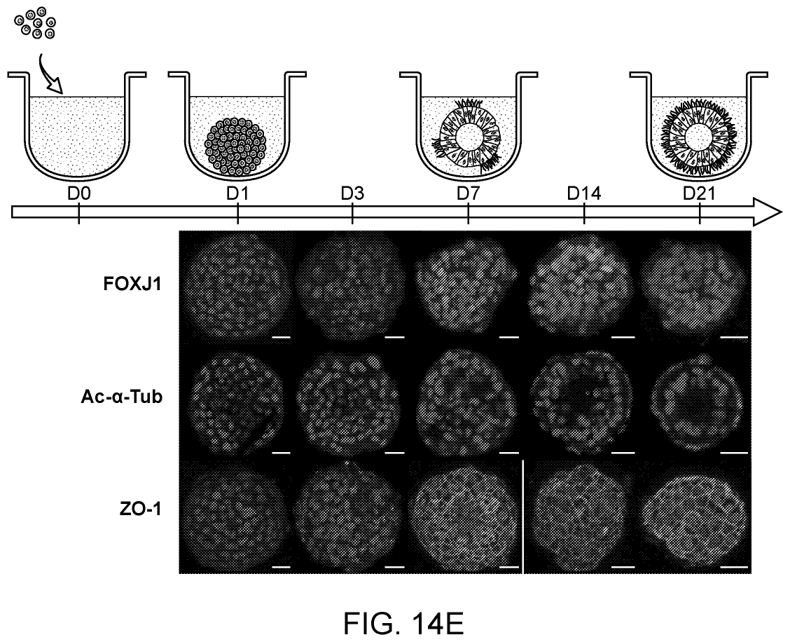

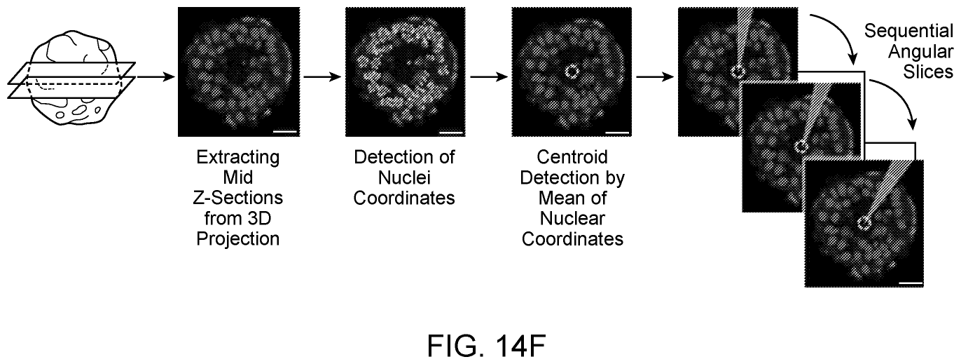

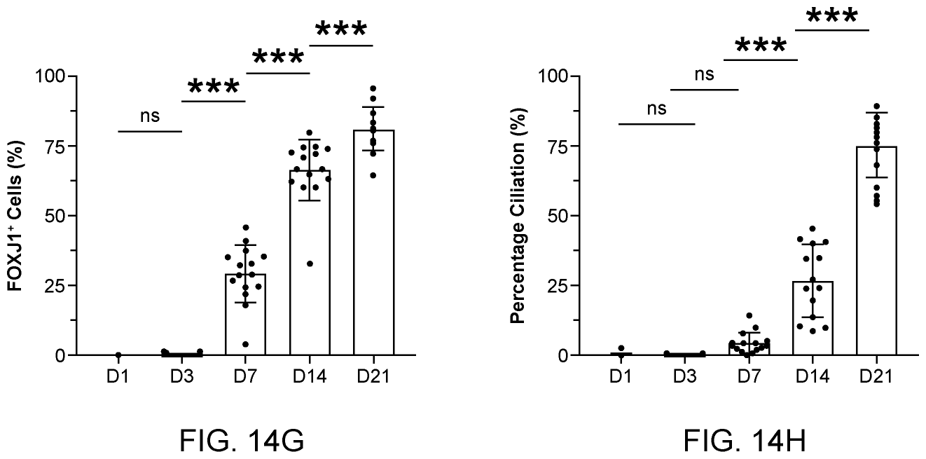

Calculating percentage ciliation in AOAOs: Z-stack images of AOAOs, stained with Acetylated-α-Tubulin (Ac-α-Tub) and DAPI, were acquired using a Zeiss LSM 700 laser scanning confocal microscope. For percentage ciliation calculations, 3 to 4 cross-sections per organoids were selected from mid-z-stacks. The pixel coordinates of the edges of DAPI stained nuclei were used to determine the centroid of the apical-out organoid. By using k means (k=1) clustering on the edge coordinates of DAPI-stained nuclei, the centroid of the organoid was determined in a robust and unsupervised manner (Virtanen et al. Nat Methods 17, 261-272, 2020). From the calculated centroid, each organoid cross-section was divided into 1-degree angular segments (FIG. 14F). Finally, the presence of Ac-α-Tub immunofluorescence signal was detected in each angular segment. The angular segments containing the α-AcTub signal were recorded and used to calculate the percentage ciliation of the organoid (Pedregosa et al., J Machine Learning Res. 12:2825-2830, 2011).

Calculating abundance of ciliated cells in AOAOs: Z-stack images of AOAOs, stained for the presence FOXJ1 using Anti-FOXJ1 antibody and for nuclei using DAPI, were acquired using a Zeiss LSM 700 laser scanning confocal microscope. For calculating the percentage abundance of FOXJ1+ ciliated cells, the mid-cross section of each organoid was selected from the z-stacks. The number of FOXJ1+ and DAPI+ cells were calculated using the Spots tool in the IMARIS software. The percentage of ciliated cells was calculated by normalizing the number of FOXJ1+ cells by the DAPI+ total cell number.

MATRIGEL© embedding of AOAOs: Mature AOAOs at day-21 to day-28 of differentiation were collected together and embedded in MATRIGEL® matrices. For MATRIGEL® embedding, collected AOAOs were resuspended in 40% (vol/vol) GFR-MATRIGEL® in differentiation medium, which was kept on a heat plate set to 37° C. for 10 minutes to enable effective gelation. Upon matrix gelation, differentiation medium was added to the top of the AOAO-containing gel matrices. All matrix-embedded AOAOs were maintained at 37° C. with 5% CO₂. The next day, 30-second video recordings of AOAOs were captured using EVOS FL Auto 2 Imaging System.

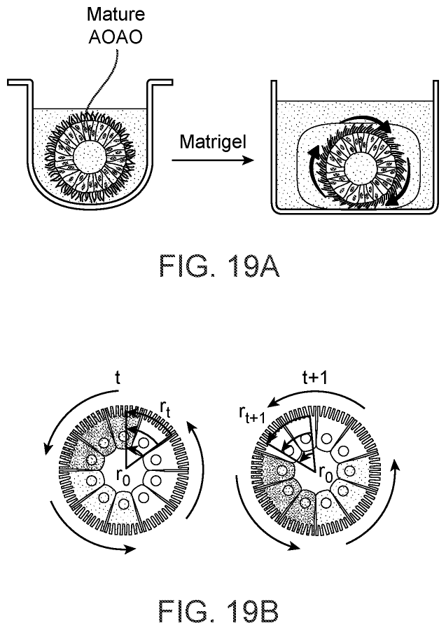

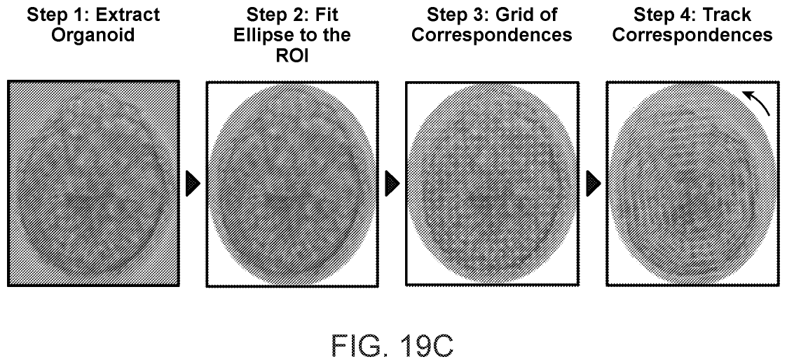

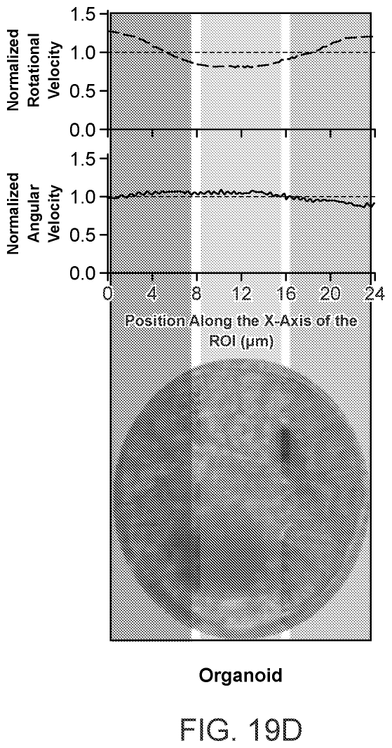

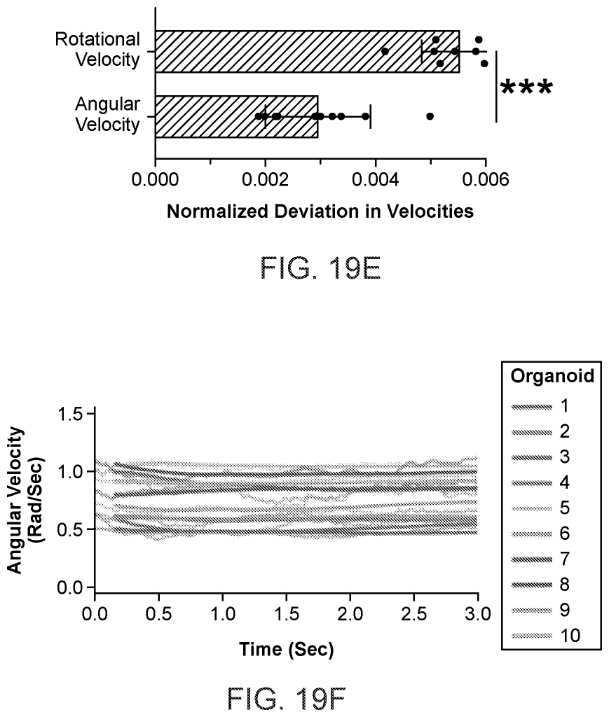

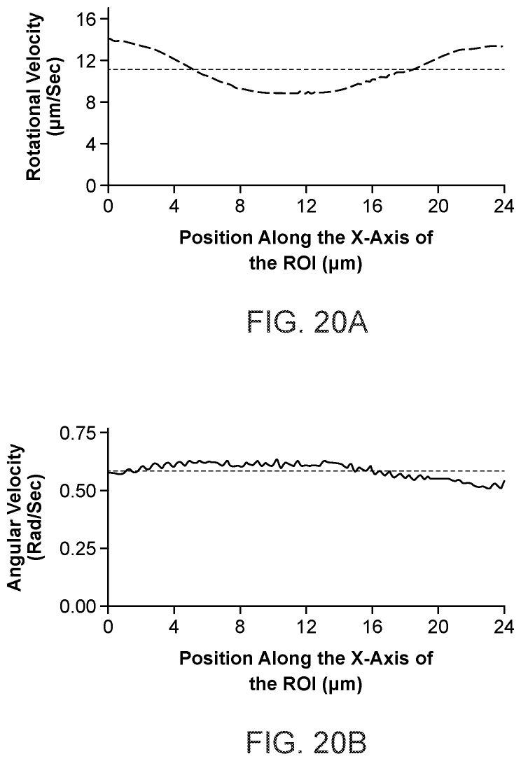

Angular velocity calculation from video data: The video recordings of AOAOs were preprocessed by cropping to the region of interest containing the organoid, using Gaussian blur to reduce the noise, and smoothing variations in contrast to improve the performance of the tracking algorithm (Ravichandran et al., View-Invariant Dynamic Texture Recognition using a Bag of Dynamical Systems. 1651-1657, 10.1109/CVPR.2009.5206847, 2009; and Lu et al., Proc. IEEE Workshop on Motion and Video Computing (WACV/MOTION'05). 241-246). Since the organoids generally had a 28 spheroid shape, an ellipse was fit to the region of interest to mask out the surrounding region of the organoid. From the first frame of the video, a grid of equispaced correspondences was selected (FIG. 19C). The correspondences were 5 pixels apart in each direction. These correspondences were tracked for the duration of the video by using the Lukas-Kanade (LK) tracking algorithm implementation from OpenCV python package (Bradski, Dr Dobb's J. Software Tools 25:120-125, 2000; Bouguet, Intel Corporation, Microprocessor Research Labs 2000; and Fu and Kovesi, in Proceedings of the 2010 International Conference on Digital Image Computing: Techniques and Applications, 468-473, IEEE Computer Society, 2010). The distance covered by each correspondence was measured and converted to the rotational velocity (Sun et al., in 2010 IEEE Computer Society Conference on Computer Vision and Pattern Recognition, 2432-2439, 2010). The correspondence that did not move, thus marking the background, was filtered out. The rotational velocity was converted to angular velocity for each organoid by dividing by the distance of correspondence from the center (FIG. 20). To eliminate the error accumulation by the LK tracking algorithm over time, the correspondences were recomputed every 25 frames. The angular velocity for each organoid was the average angular velocity of all correspondences for the entire duration of the video. The difference in rotational and angular velocity was quantified by calculating the mean normalized deviation of velocity (rotational and angular) from the mean (FIG. 19E) using the following equation:

$$Mean Squared Deviation = \frac{{( Velocity of the {position}^{2} - Mean of velocity {profile}^{2} )}^{1 / 2}}{Mean of velocity profile}$$

Treating AOAOs with Paclitaxel or EHNA for angular velocity analysis: Mature AOAOs (day-21 to day-28 of differentiation) were embedded in MATRIGEL® for two days before treatment with desired chemical inhibitors of cilia motility. For paclitaxel, AOAOs were treated with paclitaxel (20 μM, diluted in differentiation medium) for 24 hours, with the control group being treated with an equal concentration of dimethyl sulfoxide (DMSO). For EHNA, AOAOs were treated with EHNA (0.1, 0.3, or 1 mM) for 2 hours, with the control group being treated with an equal concentration of phosphate buffer saline (PBS). Following chemical treatment of desire time periods, 30-second video recordings of AOAOs, pre- and post-treatment, were captured using the EVOS FL Auto 2 Imaging System.

Imaging the AOAO cilia beating for kymographs generation and cilia beating frequency (CBF) calculation: Mature AOAOs (day-21 to day-28 of differentiation) were transferred to a 1.5-mL Eppendorf tube and kept on ice. Cold collagen type 1 was neutralized with the neutralization solution, added to the organoids at a final concentration of 2 mg/mL, and the entire AOAO-collagen mixture was transferred to the glass bottom region of a MatTek dish. The MatTek dish containing organoids in collagen was kept on ice for additional 10 minutes until the organoids settled down to the bottom of the dish. The MatTek dish was then placed on a heat plate set to 37° C. for 10 minutes. 1 mL of differentiation medium was placed in the dish before capturing video recording of cilia beating using on a Zeiss AxioObserver Z1 microscope with a 100×, 1.45 NA objective and pco.edge 5.5 camera. The high-speed video recordings of cilia beating were preprocessed using the previously described method to smoothen noise and variations in contrast. Additionally, the Contrast Limited Adaptive Histogram Equalization (CLAHE) function in Python v. 3.7 software was used to improve the contrast of the cilia with respect to the background (Lu et al., supra; and Sun et al., supra). From the preprocessed video, the region of interest on organoid surface containing cilia was cropped. The normal vector with respect to the organoid surface for each region of interest was calculated. The pixel intensity along the normal vector was then mean pooled for each frame, thereby generating the kymograph for ciliary motion. The peaks in the kymograph were counted and divided by the duration of the video to obtain the CBF value for the organoid. At least 10 kymographs were generated per organoid and the average value represented the CBF of the organoid.

Statistics: Quantitative data were displayed as means±s.d. Statistical significance was determined using one-way analysis of variance (ANOVA) with post-hoc Tukey's Test and unpaired t-test. Statistical analyses were performed using GraphPad.

### Example 5—Engineering Apical-Out Airway Organoids

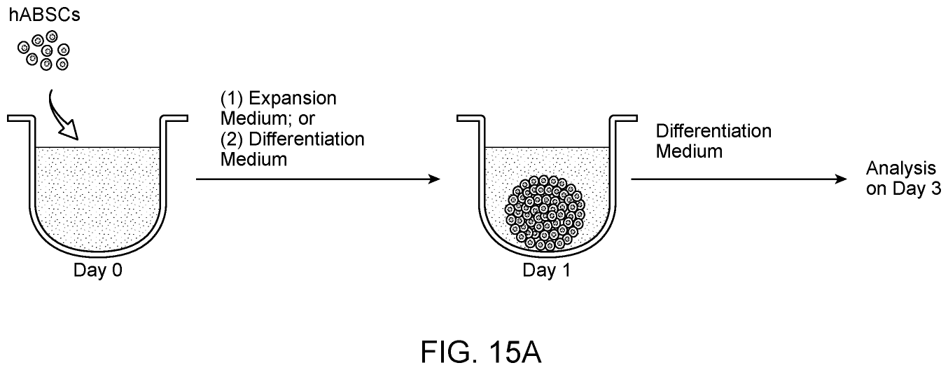

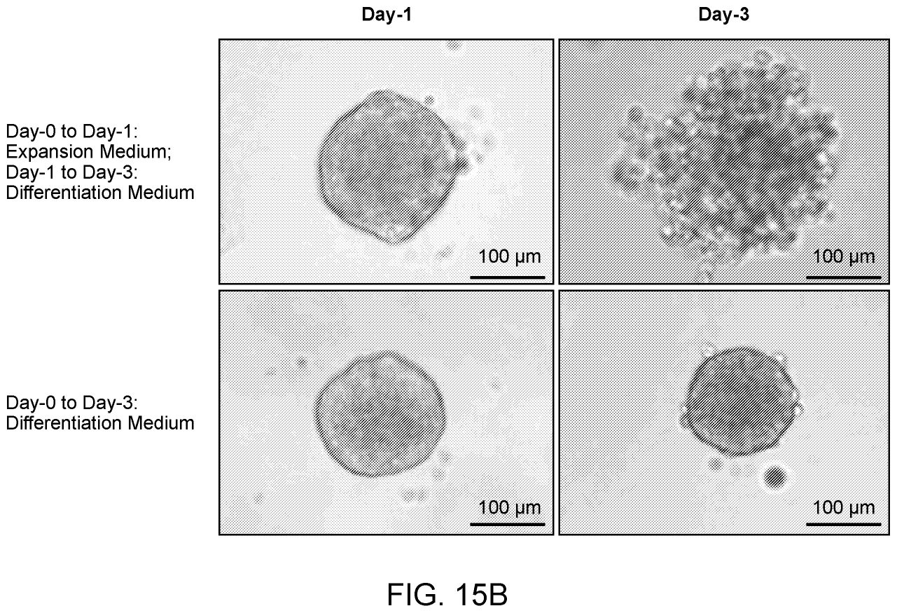

The interaction between epithelial cells and their surrounding extracellular matrix (ECM) plays instrumental roles in determining tissue polarity. Traditional apical-in organoids are typically produced from airway epithelial cells in ECM-embedded culture, leading to recognition of the organoid's exterior surface that faces the ECM to be basal-lateral and its interior surface to be apical (Rock et al., supra; McCauley et al., supra; Barkauskas et al., Development 144:986-997, 2017; Dye et al., Elife 4, doi:10.7554/eLife.05098, 2015; and Sachs et al., EMBO J 38, doi:10.15252/embj.2018100300, 2019). Studies were conducted to determine whether removal of the ECM support during airway organoid biogenesis would reverse traditional apical-basal recognition and organoid polarity (FIG. 14A). Bronchus-derived human airway basal stem cells (hABSC) were expanded in two-dimensional (2D) culture using expansion medium formulated based on BEGM™ (Levardon et al., supra). To enable airway organoid formation, a defined number (500) of hABSCs, dissociated from 3D expansion, were allowed to aggregate together on top of a cell-repellent surface in a 96-well plate with no ECM support. Following overnight suspension culture in differentiation medium (PNEUMACUL™-ALI), effective spheroid formation was observed, followed by differentiation into ciliated airway organoids with apical-out polarity (cilia beating on the outer surface) by the end of week 3. These were referred to as Apical-Out Airway Organoids (AOAOs). Compared to using expansion medium for hABSC cell aggregation followed by transitioning to differentiation medium, the use of differentiation medium for both initial cell aggregation and subsequent differentiation was essential for maintaining spheroid tissue integrity (FIG. 15).

To characterize epithelial polarity in the resulting day-21 AOAOs, immunofluorescence staining of key polarity markers of the airway epithelium was performed, and highly selective localization of ciliary Ac-α-Tub was observed on the organoid outer surface. Consistent with this orientation, epithelial tight junction protein, Zona Occludens Protein-1 (ZO-1), was observed to form highly organized intercellular junctions underneath the apical surface (FIG. 14B). Using SEM and TEM, dense motile cilia covering the organoid outer surface were observed, with typical 9+2 microtubule organization, further verifying the apical-out epithelial polarity (FIG. 14C). Next, the consistency of epithelial polarity was assessed in day-21 organoids resulting from continuous 3D suspension culture by examining Ac-α-Tub localization on the organoid's exterior versus interior surface, revealing 100% apical out polarity (FIG. 14D).

To track temporal dynamics of ciliogenesis and epithelial polarization, AOAOs were harvested on Days 1, 3, 7, 14, and 21 of suspension differentiation, and were evaluated for the ciliated cell nuclear markers Forkhead Box J1 (FOXJ1), Ac-α-Tub, and ZO-1 (FIG. 14E). FOXJ1+ ciliated cells emerged as early as Day 7, and their abundance gradually increased to 81±8% on Day 21 (FIG. 14G). The percentage ciliation was then calculated by quantifying cilia coverage on the organoid's exterior surface. To do this, mid-Z-sections were selected from confocal Z-stack images of each organoid whole mount stained for Ac-α-Tub and Hoechst-33342 (nuclei). The centroid of each Z-section was identified using k-means clustering on nuclei coordinates. Angular slices of 1-degree from the centroid were assessed regarding their overlapping with ciliary Ac-α-Tub expression on the organoid edge. Finally, the percentage ciliation was calculated by normalizing the number of angular slices containing Ac-α-Tub fluorescence signal by 360 (FIG. 14F). Applying this analytical pipeline to time series images of AOAOs, a steady increase in percentage ciliation was observed over time, reaching 76±12% on Day 21 (FIG. 14H), which echoed the gradual increase in FOXJ1+ ciliated cell abundance (FIG. 14G).

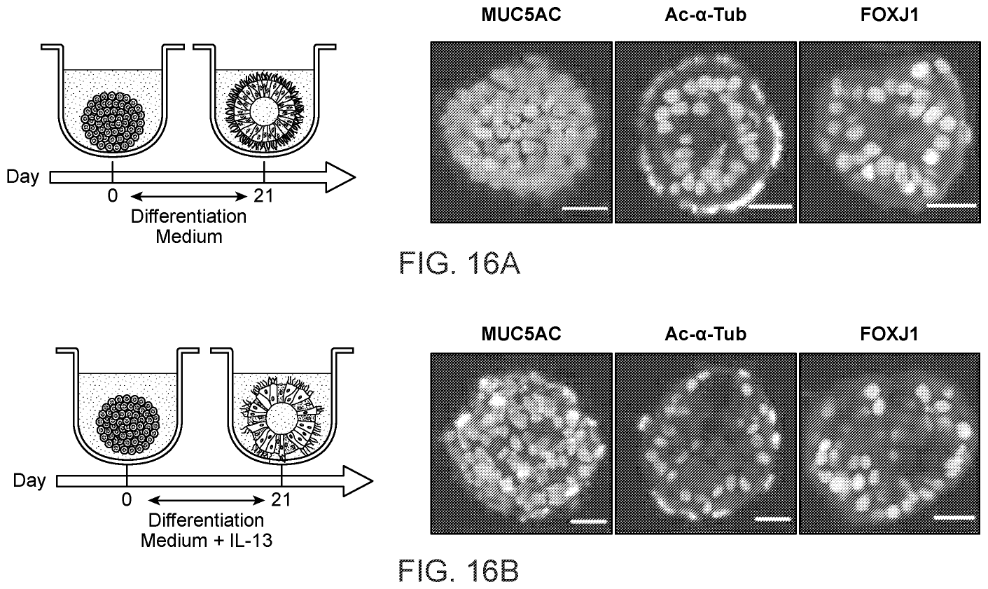

The native human airway is known to undergo goblet cell hyperplasia and mucus hypersecretion following stimulation with cytokines, such as Interleukin 13 (IL-13) (Feldman et al., Am J Respir Cell Mol Biol 61:322-331, 2019). In AOAOs engineered using standard differentiation medium, no MUC5AC+ goblet cells was observed on Day 21. In sharp contrast, when IL-13 (5 ng/mL) was supplemented to the differentiation medium, massive induction of goblet cells was observed in Day 21 AOAOs (FIG. 16).

### Example 6—Assessing Reversibility of AOAO Epithelial Polarity

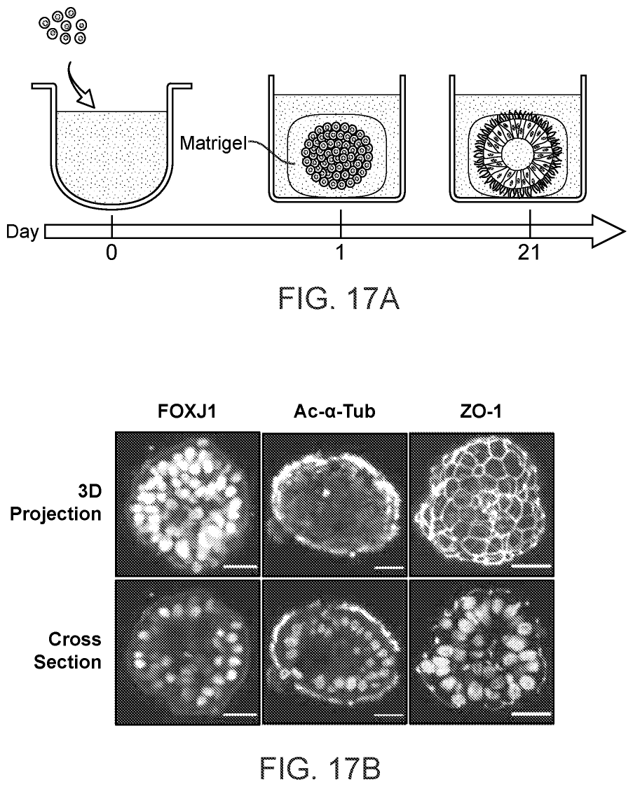

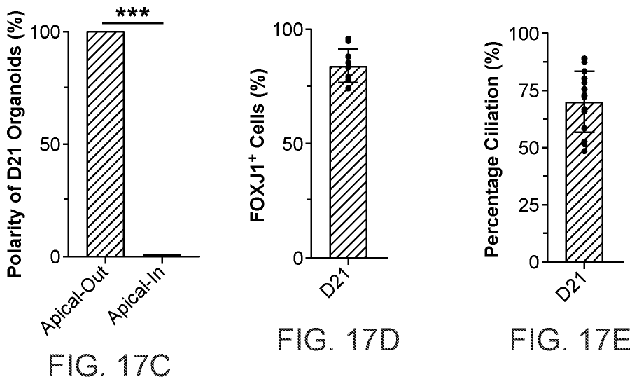

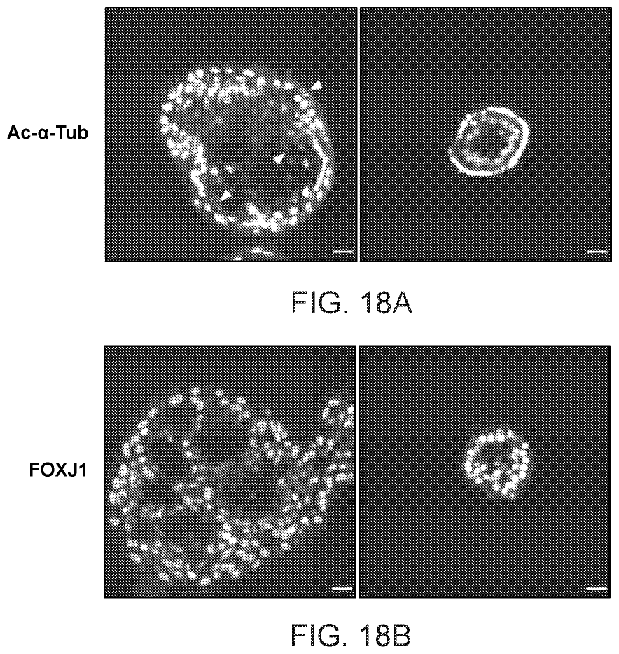

Upon demonstrating the ECM-free suspension culture as a driver for establishing consistent apical-out airway polarity, the stability of such epithelial polarity was investigated when the surrounding extracellular environment changed. To do this, hABSC aggregates were transitioned, following only 1-day of suspension culture, into MATRIGEL®-embedded culture and continued the differentiation until Day 21. Surprisingly, as indicated by FOXJ1, Ac-α-Tub, and ZO-1 expression (FIG. 17A), all organoids subjected to this two-phase culture procedure (1 day in suspension followed by 20 days in MATRIGEL® embedding) still exhibited homogenous apical-out polarity (FIGS. 17B and 17C). Further, these organoids from two-phase culture underwent robust ciliogenesis leading to Day 21 ciliated cell abundance (83±7% FOXJ1+; FIG. 17D) and percentage ciliation (70±14%; FIG. 17E) that was not statistically different from that of standard AOAOs that had only experienced suspension culture (p=0.2964 for ciliated cell abundance; p=0.1448 for percentage ciliation). These results implied that airway epithelial polarity was effectively established within the first day of 3D suspension culture and remained stable even after being transitioned to ECM186 supported culture. During the transition from suspension to MATRIGEL®-embedded culture, sporadic merging of individual hABSC aggregates into larger organoid bodies also was observed, where Ac-α-Tub expression was found on both the interior and exterior surfaces (FIG. 18).

### Example 7—Developing Computer Vision Algorithms to Assess AOAO Rotation

Intriguingly, the beating motion of exterior facing cilia endowed motility to the AOAO, which exhibited random movement in suspension culture. The possibility of stabilizing such cilia-powered AOAO motility was investigated by providing a 3D surrounding material support for cilia to propel against. To do this, mature AOAOs (between Day 21 and Day 28 of suspension differentiation) were embedded within a MATRIGEL® matrix (FIG. 19A), which effectively enabled the AOAOs to adopt a stable rotational motion, offering a unique opportunity to transform nanoscale, high-frequency cilia motility into microscale, low-frequency organoid rotation.

To reliably quantify the rotational motion of AOAOs, computer vision-based motion tracking algorithms were developed (Bradski, Dr Dobbs J. Software Tools 25:120-125, 2000; and Bouguet, Intel Corporation, Microprocessor Research Labs, 2000). From video recordings of AOAO rotation, the center of each organoid (r0) and the position of the correspondence being tracked (rt) were detected, and these vectors were used to determine the distance of the correspondence from the center. The change in position of correspondence (rt+1) was used in the next step to calculate the distance covered by the correspondence (FIG. 19B). To quantify the rotational motion, the region of interest (ROI) was identified by fitting an ellipse to the organoid to suppress the surrounding background. A grid of correspondences in the ROI was generated, and the correspondences were then tracked by the tracking algorithm. The distance covered by correspondences was then divided by the time taken to determine rotational velocity (FIG. 19C).

The rotational velocity calculated above was dependent on the distance of the correspondence being tracked from the AOAO center. This led to large variation in measurements obtained at different regions of the same organoid. For example, the organoid's rotational velocity profile had a parabolic shape with minimum at the central region and maximum at the periphery (FIGS. 19D and 20A). This was due to the correspondences close to the organoid center not covering a large distance in comparison to those at the periphery, which as a result yielded a lower rotational velocity. To overcome this constraint, the angular velocity of each correspondence, which became independent on its exact position within the organoid, was calculated by dividing the rotational velocity by the distance of each correspondence from the organoid center (FIG. 20B). The angular velocity of the entire AOAO was determined by taking the mean of the angular velocity of all the correspondences being tracked. To compare the rotational and angular velocity profiles across the entire length of the AOAO, the mean squared deviation of the velocity was calculated from its mean value and then normalized by the mean value (FIG. 19E). The deviation in rotational velocity was 2-fold greater than that in angular velocity. Therefore, to ensure consistency in measuring AOAO rotational motion, the angular velocity was utilized as the main readout. Finally, to detect the time-dependent variability in tracking AOAO rotation, the instantaneous angular velocity of 10 representative AOAOs was visualized. The running mean of instantaneous angular velocity showed consistent rotational motion for AOAO throughout the entire recorded time period (FIG. 19F).

### Example 8—Assessing Drug-Induced Inhibition of Cilia Motility and AOAO Rotation

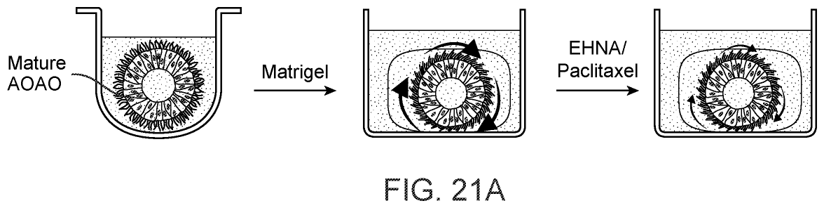

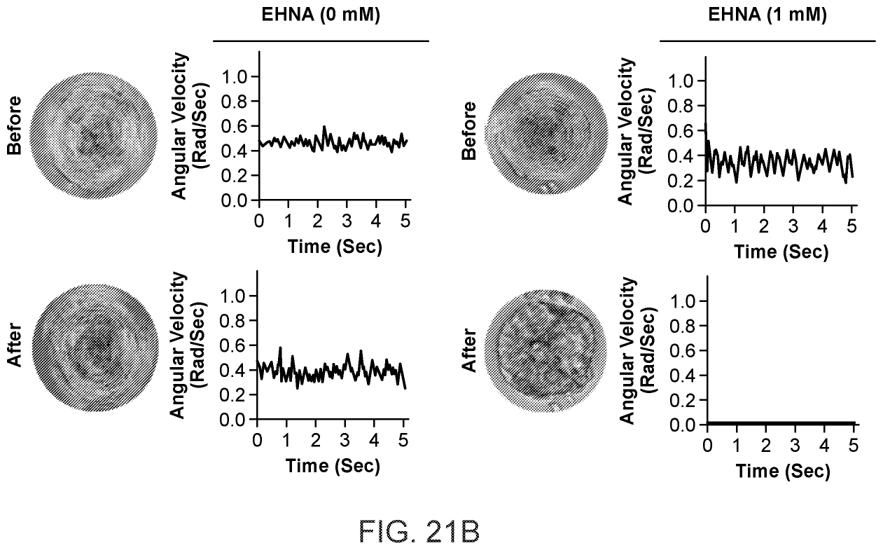

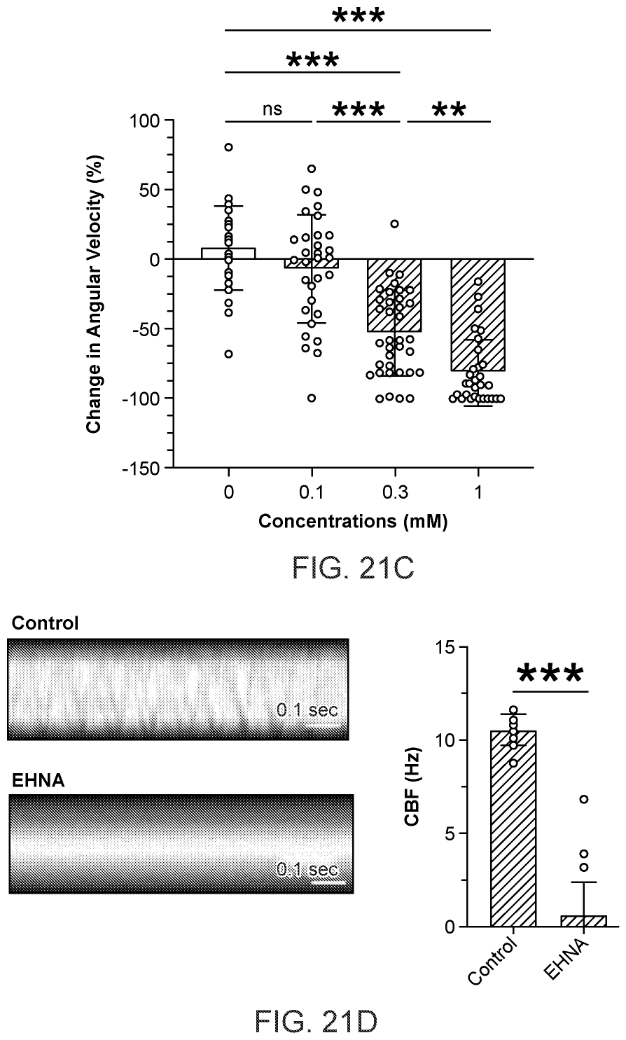

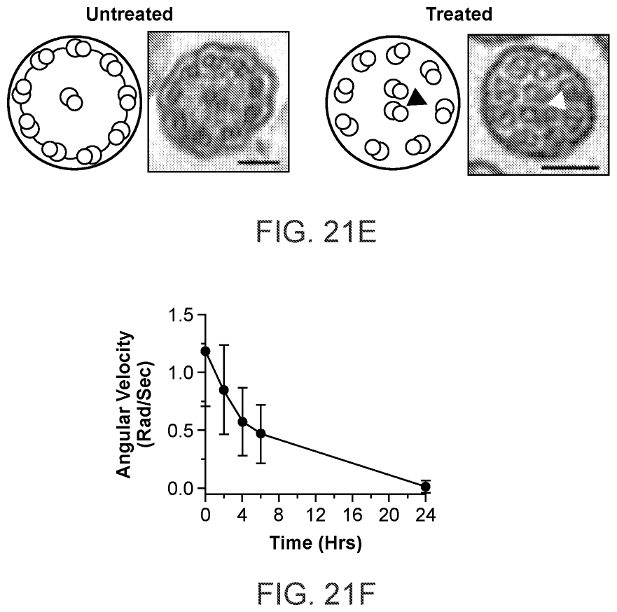

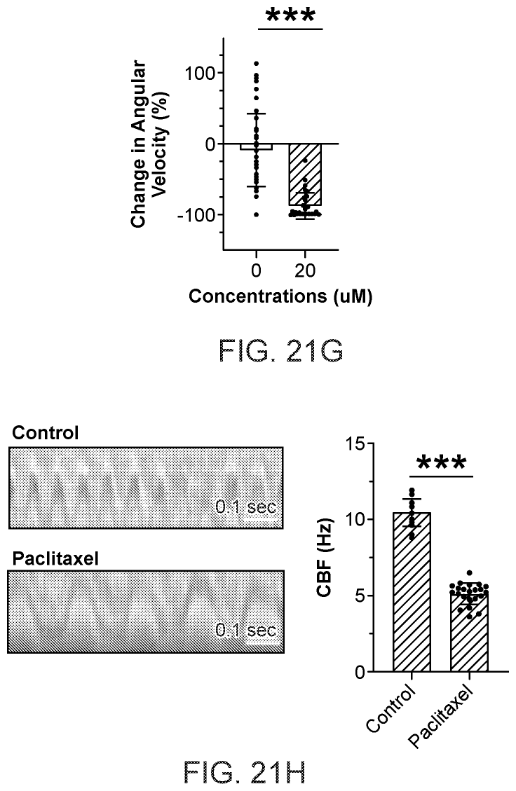

To assess the correlation between cilia motility and cilia-powered AOAO rotational motion, known chemical inhibitors of cilia motility were applied to MATRIGEL®-embedded, mature AOAOs (FIG. 21A). EHNA (erythro-9-(2-hydroxy-3-nonyl)adenine) is an inhibitor of dynein, the molecular motor that powers axonemal doublet microtubule sliding and thus cilia beating (Schliwa et al., Proc Natl Acad Sci USA 81:6044-6048, 1984; and Bouchard et al., Proc Natl Acad Sci USA 78:1033-1036, 1981). The computer vision-based motion tracking algorithms developed as described above were used to compute the angular velocity of the same organoid before and after EHNA treatment (FIG. 21B). EHNA was introduced at a range of concentrations (0, 0.1, 0.3 and 1 mM) to mature AOAOs for 2 hours, and an EHNA-dose-dependent reduction in organoid angular velocity was observed (FIG. 21C). In parallel, the inhibitory effect of EHNA on cilia beating frequency (CBF) was confirmed using kymography analysis (FIG. 21D).

Paclitaxel is a chemotherapeutic agent that stabilizes microtubule structures and thus interferes with microtubule-dependent mitosis, cell migration and cilia beating (Zhu and Chen, Cell Mol Biol Lett 24:40, 2019; Orr et al., Oncogene 22:7280-7295, 2003; and Schiff et al., Nature 277:665-667, 1979). Treatment of mature AOAOs with paclitaxel (20 μM) for 24 hours led to abnormalities in ciliary ultrastructure (FIG. 21E), which were in line with studies described elsewhere (Boisvieux-Ulrich et al., J Cell Sci 92(Pt 1):9-20, 1989; and Shinohara et al., Dev Cell 35:236-246, 2015). The MATRIGEL®-embedded AOAOs were then incubated with paclitaxel (20 μM), and were monitored periodically for 24 hours, revealing paclitaxel-induced, progressive reduction of organoid angular velocity (FIGS. 21F and 21G). Consistent with this, 24-hour paclitaxel treatment dramatically reduced CBF shown by kymography analysis (FIG. 21H). These findings validated the idea that the angular velocity of the AOAO correlates with and predicts cilia motility.

### Example 9—Modeling and Characterization of Genetic Ciliopathy Using AOAOs

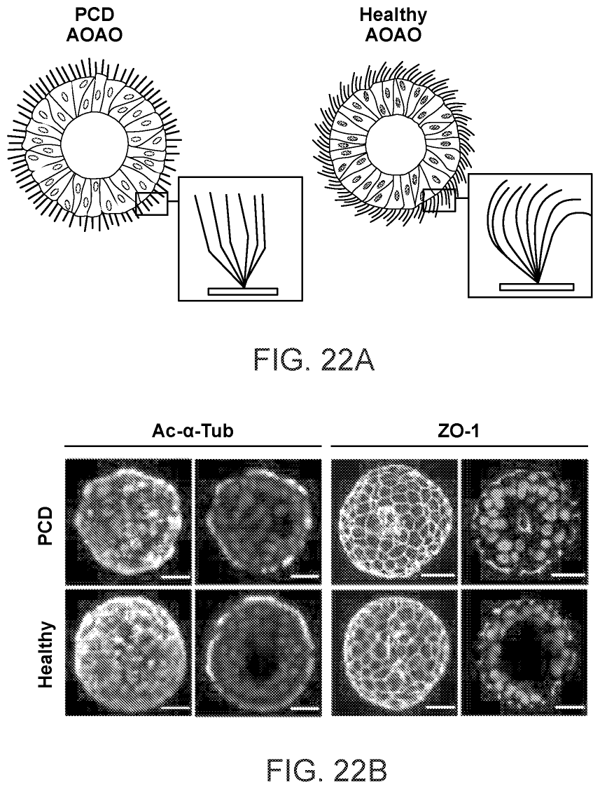

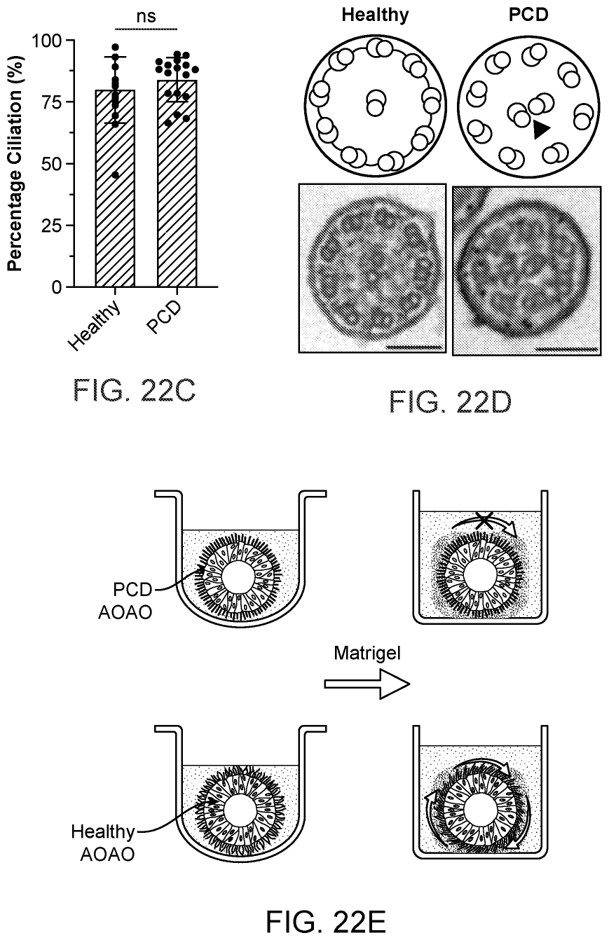

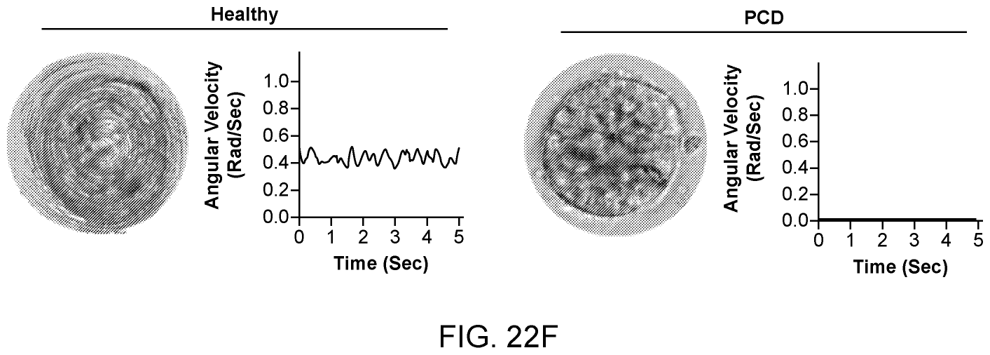

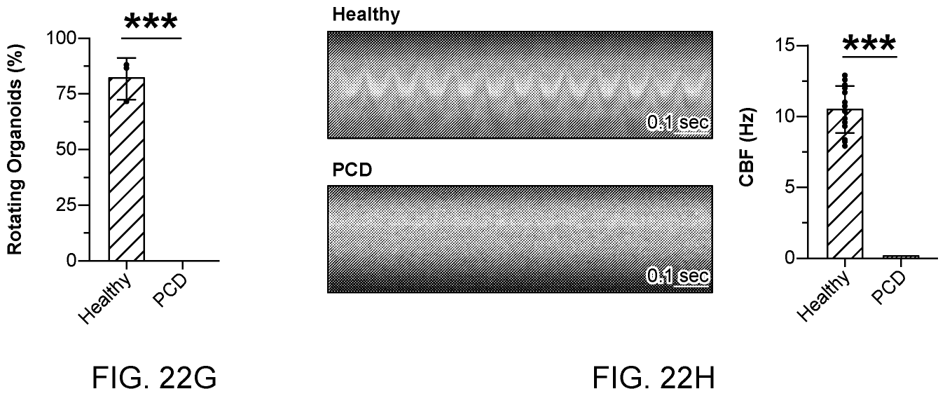

Primary ciliary dyskinesia (PCD) is a collection of genetic disorders involving abnormal motile cilia ultrastructure and function (Antony et al., Hum Mutat 34:462-472, 2013; Blanchon et al., J Med Genet 49:410-416, 2012; Dutcher et al., Am J Respir Cell Mol Biol 62:281-282, 2020; Horani et al., Paediatr Respir Rev 18:18-24, 2016; and Brennan et al., Int J Pediatr Otorhinolaryngol 142:110586, 2021). Mutations in the CCDC39 gene cause inner dynein arm defects and axonemal disorganization in cilia, and have been associated with PCD (Blanchon et al., supra). Using hABSCs carrying mutations in the CCDC39 gene, studies were conducted to assess whether AOAOs would be effectively generated from PCD-bearing cells, and whether the PCD-associated ciliary defects would be recapitulated by the AOAO rotational motion. hABSCs isolated from healthy and PCD (with CCDC39 mutations) patients were expanded and transitioned for AOAO formation via 3D suspension culture (FIG. 22A). Following 21 days of differentiation in suspension, as indicated by Ac-α-Tub and ZO-1 expression, airway organoids engineered from both healthy and PCD cells underwent effective epithelial differentiation with consistent apical-out polarity (FIG. 22B). Further, comparable percentage ciliation was observed on the apical surface of healthy and PCD organoids (p=0.3212), indicating that the CCDC39 mutations did not affect basic ciliogenesis (FIG. 22C). However, as expected, PCD organoids exhibited defects in ciliary ultrastructure as indicated by TEM, showing a surrounding microtubule pair being mislocated to the center, compared to the normal 9+2 ciliary ultrastructure observed in healthy organoids (FIG. 22D). Building on these morphological findings, studies were conducted to assess and compare the rotational motion of PCD and healthy AOAOs by transferring them, following maturation, from 3D suspension culture to MATRIGEL® embedding (FIG. 22E). Consistent with defective ciliary structures, none of the embedded PCD AOAOs were able to rotate, as compared to over 75% of the embedded healthy AOAOs showing stable rotational motion (FIGS. 22F and 22G). Lastly, CBF analysis was performed, revealing that obvious cilia motility was not observed in PCD organoids as compared to robust cilia beating in healthy organoids (FIG. 22H). These findings further validated the AOAO model and its associated computational analysis pipeline as effective tools for modeling and assessing human motile ciliopathy.

### Example 10—Using Motile AOAOs to Investigate Respiratory Injuries

Industrialization and urbanization have heavily contributed to increased sources of pollutants, making air pollution a key public health issue. Particulate matter (PM) that causes major respiratory health threats include car exhaust emissions, industrial emissions, smoke, burn, and dust. Diesel particulate matter (DPM; Standard Reference Material 1650b, National Institute of Standards and Technology) is one of the major solid toxic particulates in exhaust emitted into the air by diesel engines, and can lead to inflammation and reduced lung function.

Studies were conducted to establish the human AOAO as a model system for assessing pollution particulate-induced respiratory injuries to mature airway epithelium and regeneration, using DPM as the source of injury. In particular, enabled by the apical-out organoid polarity, both pollutant administration and injury assessment were performed in a non-invasive manner, overcoming the limitations of previously used apical-in airway models (McCauley et al, supra; and Rock et al., supra).

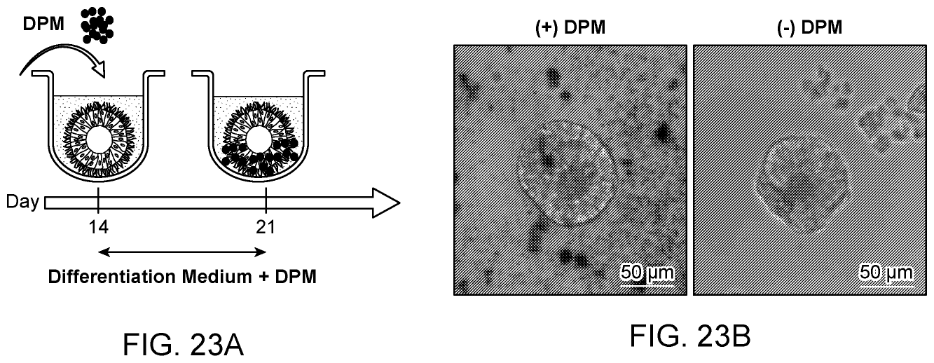

In particular, a first set of studies were conducted to assess pollution particulate-induced respiratory injuries to mature airway epithelium. Human airway epithelium has the capability of self-repair following injuries. Many airborne pollutants not only cause the initial injuries but also impair subsequent epithelial repair, worsening the impact of air pollutants on respiratory health (Butz and Kim, Physiol. Behav. 176:139-148, 2017). DPM-induced injury to mature airway epithelium was investigated by treating Day 14 AOAOs with DPM (200 μg/mL) (Thomas et al., Environ. Sci. Pollut. Res. 2:69-72, 1995; and Lee et al., Biomolecules. 11:1-15, 2021) for 7 days (FIGS. 23A and 23B). To do this, effective DPM homogenization methods that combined bovine serum albumin coating and sonication were established, enabling consistent treatment of AOAOs with DPM.

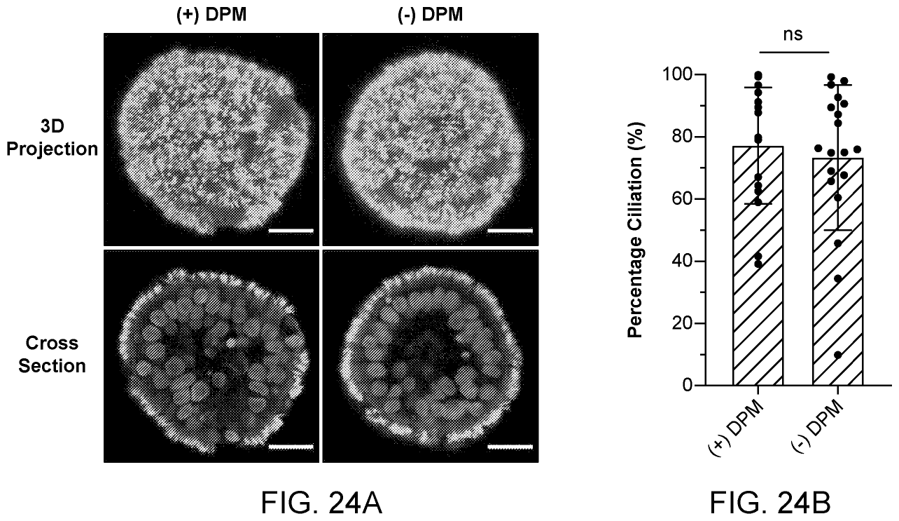

Mucus hypersecretion is commonly associated with pollution-induced respiratory injuries (Cooper and Loxham, Eur Respir Rev. 28(153):190066, 2019; and Wang et al., Cell. Signal. 53:122-131, 2019), which together with cilia beating defects, leads to impaired mucociliary clearance and breathing difficulty. To assess DPM-induced ciliary defects in the AOAOs, immunofluorescence staining of the cilia marker acetylated-alpha-tubulin (Ac-α-Tub) was performed, and the percentage ciliation was calculated using localization of ciliary Ac-α-Tub on the organoid outer surface and previously described percentage ciliation analytical pipeline (Example 5; FIGS. 24A and 24B). These studies demonstrated that AOAOs treated with DPM and control AOAOs showed no statistically significant difference in percentage ciliation, indicating that DPM had no direct effect on cilia differentiation in Day 14 mature AOAO epithelium.

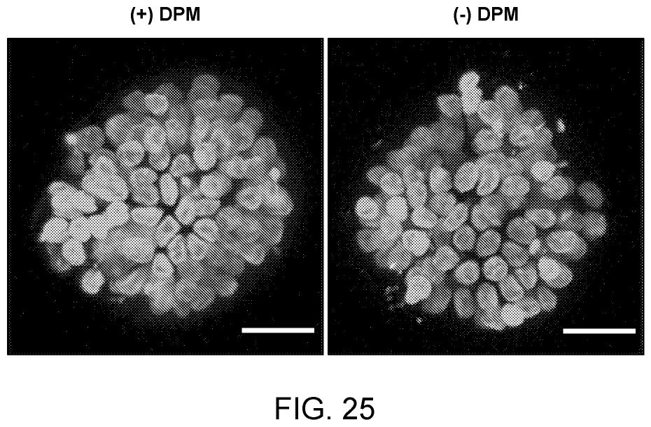

DPM-induced mucus-hypersecretion in the mature epithelium of AOAO also was assessed via DPM treatment during Day 14 to Day 21. Similar to cilia differentiation, DPM had no inflammatory effect on the mature airway epithelium, and exhibited no sign of goblet cell hyperplasia or mucus hypersecretion, as evident from immunofluorescence staining for MUC5AC+ goblet cells (FIG. 25).

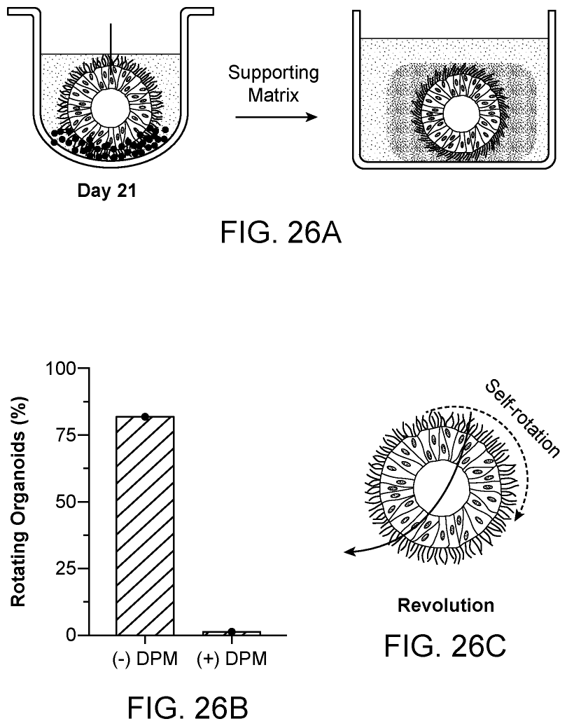

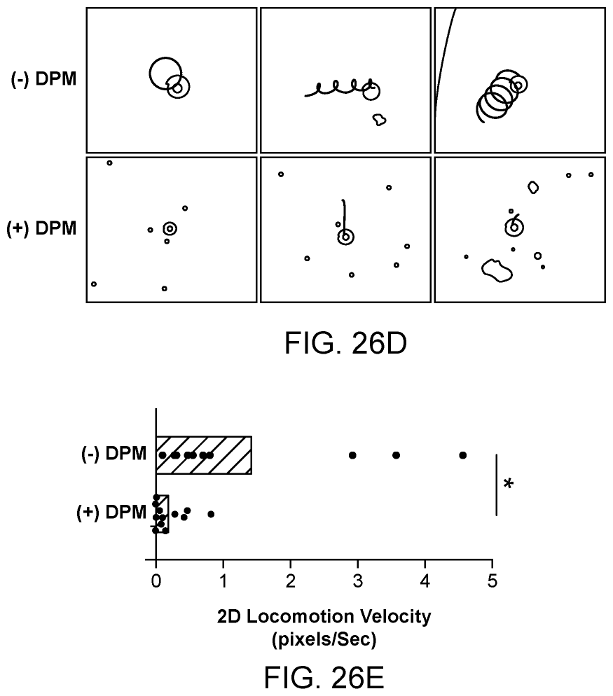

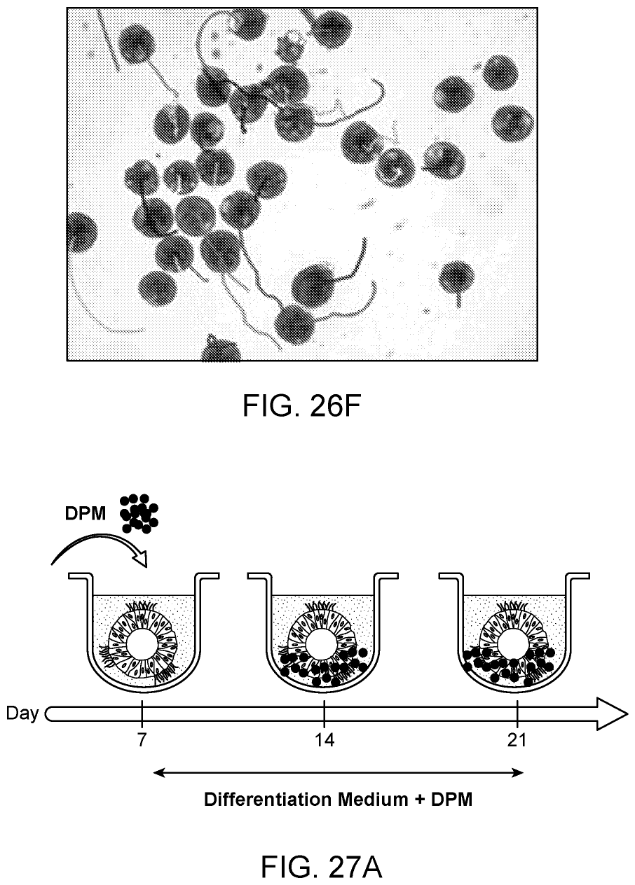

Cilia motility on the exterior surface of the AOAO drives organoid rotation when embedded in a supporting matrix. To further assess the effects of DPM on the differentiation and beating of cilia as evidenced by AOAO rotational motion, AOAOs were transferred, after Day 14 to Day 21 DPM treatment, from 3D suspension culture to MATRIGEL® embedding (FIG. 26A). None of the embedded DPM-treated AOAOs were able to rotate, as compared to over 75% of the embedded control AOAOs that showed stable rotational motion (FIG. 26B). These findings implied that DPM indeed impaired cilia motility even though it had no direct impact on cilia differentiation. These findings also further validated the AOAO model as an effective tool for assessing human motile ciliopathy with great sensitivity to external environmental agents, similar to the native airway.

The beating of the exterior-facing cilia in AOAOs confers coordinated locomotion on a 2D surface, which is characterized by a simultaneous revolutionary and self-rotary motion of the AOAO (FIG. 26C). While the 3D organoid rotation assay offers an effective means for assessing human motile ciliopathy, it requires 3D hydrogel embedding. Therefore, utilizing the AOAO locomotion, a Computer Vision (CV)-based framework was utilized to assess the organoid migration on a 2D surface without the need for hydrogel embedding, transforming organoid cilia motility assessment into a simple, convenient, and high-throughput analysis. The CV-based framework allowed for tracking of the organoids in a captured video to perform real-time analysis via optical flow tracking. The CV framework improved the interpretability of the videos by using contrast limited adaptive histogram equalization (CLAHE). From the video, correspondences (pixels) were selected and subsequently tracked by optical flow. Optical flow computed the displacement of the pixels of each image at time t and time t+dt. Optical flow was based on the optical flow tracking output to calculate the features to describe the organoid motion in the captured videos, such as eccentricity of circular motion, pitch of helical motion, and velocity. While the CV framework extracted relevant information of organoid locomotion, the automated nature of this framework allowed for effective processing of large datasets of organoid videos.

The cilia beating defects induced by DPM were confirmed by analysis of AOAO 2D locomotion following DPM treatment during Days 14 to 21 of AOAO culture (FIG. 26D). Here, the locomotion velocities of DPM treated AOAOs were significantly reduced compared to locomotion velocities of control organoids, which validated the 3D organoid rotation assay finding in which DPM had negative effects on cilia beating behavior (FIG. 26E). The CV-based framework also was applied to generate the trajectories of multiple AOAOs in a video simultaneously, in order to assess AOAO locomotion and interactions as a population (FIG. 26F). Taken together, these findings proved the potential of these technologies for accelerating the assessment of cilia motility and respiratory injury.

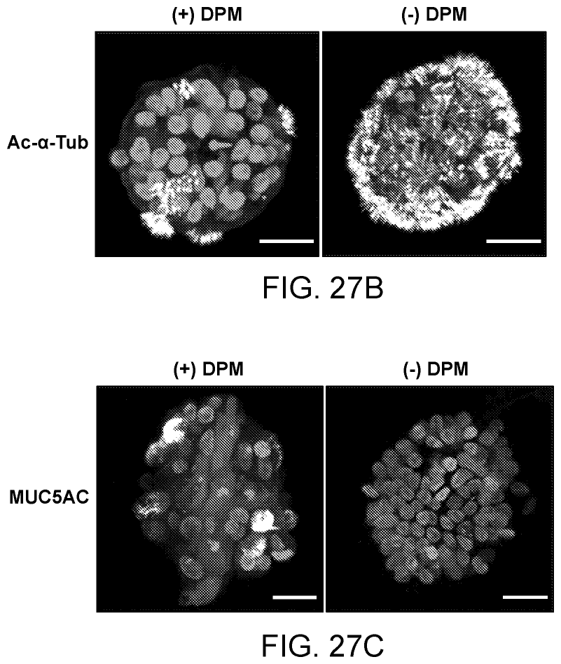

Further studies were conducted to evaluate pollution particulate-induced injury effects on airway stem cell differentiation and regeneration. Airway remodeling regularly occurs as a response to injury leading, to altered airway morphology and function (Fehrenbach et al., Cell Tissue Res. 367:551-569, 2017). The healthy pseudostratified airway epithelium has a minimum number of basal stem cells that become activated and transition themselves from the self-renewing phase to the differentiation phase in order to orchestrate regeneration and maintain the epithelial barrier during injury (Butz and Kim, supra; and Basil et al., Cell Stem Cell. 26:482-502, 2020). Here, to investigate DPM-induced injury to airway stem cell differentiation and epithelial regeneration, the early-stage epithelium of day-7 AOAOs was treated with DPM (200 μg/mL) (Thomas et al., supra; and Lee et al., supra) for 14 days during their differentiation into pseudostratified epithelium (FIG. 27A).

Airway regeneration was assessed following DPM-induced airway injury, using the ciliated cell and goblet cell lineage-specific markers Ac-α-Tub and MUC5AC, respectively. A significant reduction in ciliated cells was observed in the AOAOs that were subjected to 14 days of DPM treatment as compared to control AOAOs, indicating the impact of DPM on cilia differentiation in AOAOs (FIG. 27B). The DPM-treated early-stage epithelium in AOAOs also was analyzed for the goblet cell marker MUC5AC by immunostaining, revealing a massive induction of goblet cells (FIG. 27C). These results demonstrated that DPM was responsible for triggering inflammatory responses in the AOAOs as evidenced by the increase in goblet cells and the reduction in ciliated cells, which had an overall effect on airway basal stem cell differentiation and regeneration.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for determining whether an agent has an effect on a ciliated organoid having apical-out polarity, the method comprising:
contacting a ciliated organoid with an agent,
comparing a characteristic of the organoid after the contacting with the same characteristic of the organoid before the contacting, and
when the characteristic after the contacting has changed as compared to the characteristic before the contacting, determining that the agent has an effect on the organoid, or when the characteristic after the contacting has not changed as compared to the characteristic before the contacting, determining that the agent does not have an effect on the organoid.

2. The method of claim 1, wherein the ciliated organoid comprises airway epithelial cells, fallopian tube epithelial cells, middle ear epithelial cells, or brain ventricular epithelial cells.

3. The method of claim 2, wherein the ciliated organoid is an apical-out airway organoid (AOAO) that comprises normal human bronchial epithelial cells (NHBEs), tracheal epithelial cells, nasal epithelial cells, or any combination thereof.

4. The method of claim 2, wherein the ciliated organoid further comprises stromal cells, vascular endothelial cells, immune cells, or any combination thereof.

5. The method of claim 1, wherein the characteristic comprises coordinated percentage ciliation, ciliary beating, goblet cell specification, organoid rotation, organoid angular velocity, organoid locomotion in two dimensions, mucus secretion, cytokine secretion, extracellular matrix secretion, cell viability, or cell death.

6. The method of claim 1, wherein the characteristic comprises coordinated ciliary beating, organoid rotation in three dimensions, or organoid rotation in two dimensions.

7-8. (canceled)

9. The method of claim 1, wherein the agent is a therapeutic agent, a pathogen, a pollutant, or a chemical or biological agent.

10-12. (canceled)

13. The method of claim 9, wherein the chemical or biological agent is a cytokine or a cytotoxic reagent.

14. The method of claim 1, wherein the agent is radiation.

15. The method of claim 1, comprising determining the characteristic about 1 hour to about 28 days after the contacting.

16. A method for producing a ciliated organoid having apical-out polarity, the method comprising:
suspending a plurality of epithelial cells in a differentiation medium supplemented with a cytoskeletal structure modulator, wherein the medium does not contain extracellular matrix components,
placing an aliquot of the suspended cells into one or more wells of a cell-repellent microplate, and
maintaining the microplate for 14 to 28 days under conditions such that the suspended cells aggregate and differentiate to form a ciliated organoid with apical-out polarity.

17. The method of claim 16, wherein the epithelial cells comprise airway basal cells, fallopian tube epithelial cells, middle ear epithelial cells, brain ventricular epithelial cells, or any combination thereof.

18. The method of claim 17, wherein the epithelial cells comprise airway basal cells, and wherein the airway basal cells comprise normal human bronchial epithelial cells (NHBEs), airway bronchial stem cells (ABSCs), tracheal epithelial cells, nasal epithelial cells, or any combination thereof.

19. The method of claim 16, further comprising adding stromal cells, vascular endothelial cells, immune cells, or any combination thereof to the plurality of epithelial cells.

20. The method of claim 16, wherein the differentiation medium is an air-liquid interface (ALI) medium.

21. The method of claim 16, wherein the cytoskeletal structure modulator is a Rho-associated kinase (ROCK) inhibitor.

22. The method of claim 21, wherein the ROCK inhibitor is Y27632.

23. The method of claim 16, wherein the aliquot contains about 50 to 5000 cells.

24. The method of claim 16, wherein the cell-repellant microplate is a 96-well plate.

25. The method of claim 24, wherein the 96-well plate has U-shaped wells or V-shaped wells.

26. (canceled)

27. The method of claim 16, wherein the cell-repellant microplate is a 12-well, 24-well, 48-well, 384-well, or 1536-well plate.

28. The method of claim 16, wherein the conditions comprise a temperature of about 37° C. and an atmosphere containing 5% CO₂.
